(19) United States
(12) Patent Application Publication
(10) Pub. No.: US 2007/0249630 A1
(43) Pub. Date: Oct. 25, 2007

(54) Phenethanolamine derivatives for treatment of respiratory diseases

(75) Inventors: Keith BLAKE, Dartford (GB); Diane Mary COE, Stevenage (GB); Panayiotis Alexandrou PROCOPIOU, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: GLAXO GROUP LIMITED, Greenford (GB)

(21) Appl. No.: 11/766,879

(22) Filed: Jun. 22, 2007

Related U.S. Application Data
(63) Continuation of application No. 10/506,173, filed on Aug. 27, 2004, now abandoned.

(30) Foreign Application Priority Data
Feb. 28, 2002 (GB) 0204719

(51) Int. Cl. A61K 31/195; A61K 31/4164; A61K 31/44; A61P 11/00; C07C 63/00; C07D 239/02

(52) U.S. Cl. 514/256; 514/357; 514/396; 514/567; 540/2; 544/242; 546/329; 548/300.1; 562/405

(57) ABSTRACT

The present invention relates to novel compounds of formula (I), to a process for their manufacture, to pharmaceutical compositions containing them, and to their use in therapy, in particular their use in the prophylaxis and treatment of respiratory diseases.

The present invention is concerned with phenethanolamine derivatives, processes for their preparation, compositions containing them and their use in medicine, particularly in the prophylaxis and treatment of respiratory diseases.

Certain phenethanolamine compounds are known in the art as having selective stimulant action at β₂-adrenoreceptors and therefore having utility in the treatment of bronchial asthma and related disorders. Thus GB 2 140 800 describes phenethanolamine compounds including 4-hydroxy-α¹-[[[6-(4-phenylbutoxy)hexyl]amino]methyl]-1,3-benzenedimethanol 1-hydroxy-2-naphthalenecarboxylate (salmeterol xinafoate) which is now used clinically in the treatment of such medical conditions.

Although salmeterol and the other commercially available β₂-adrenoreceptor agonists are effective bronchodilators, the maximum duration of action is 12 hours, hence twice daily dosing is often required. There is therefore a clinical need for compounds having potent and selective stimulant action at β₂-adrenoreceptors and having an advantageous profile of action.

British Patent Application No 2,159,151 describes phenethanolamine compounds of the general formula 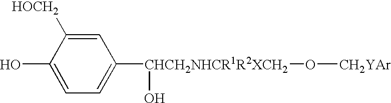
wherein Ar represents a phenyl group optionally substituted by one or more substituents selected from halogen atoms, or C₁₋₆alkyl, —(CH₂)_{q}R, [where R is hydroxy, C₁₋₆alkoxy, —NR³R⁴ (where R³ and R⁴ each represents a hydrogen atom, or a C₁₋₄alkyl group, or —NR³R⁴ forms a saturated heterocyclic amino group which has 5-7 rings members and optionally contains in the ring one or more atoms selected from —O— or —S— or a group —NH— or —N(CH₃)—),
—NR⁵COR⁶ (where R⁵ represents a hydrogen atom or a C₁₋₄alkyl group, and R⁶ represents a hydrogen atom or a C₁₋₄alkyl, C₁₋₄alkoxy, phenyl or —NR³R⁴ group), —NR⁵SO₂R⁷ (where R⁷ represents a C₁₋₄alkyl, phenyl or —NR³R⁴ group), —COR⁸ (where R⁸ represents hydroxy, C₁₋₄alkoxy or —NR³R⁴), —SR⁹ (where R⁹ is a hydrogen atom, or a C₁₋₄alkyl or phenyl group), —SOR⁹, SO₂R⁹, or —CN, and q represents an integer from 0 to 3], —O(CH₂)ᵣR¹⁰ [where R¹⁰ represents a hydroxy or C₁₋₄alkoxy group and r is an integer 2 or 3], or —NO₂ groups or an alkylenedioxy group of formula —O(CH₂)pO—, where p represents an integer 1 or 2.

We have now found that a particular group of compounds, some of which represent a selection from the broad disclosures of GB 2,159,151, have advantageous properties as will be described in more detail below.

According to the present invention, there is provided a compound of formula (I) 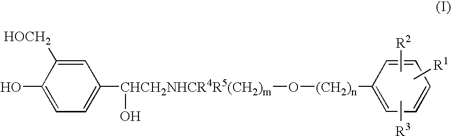
or a salt, solvate, or physiologically functional derivative thereof, wherein:
m is an integer of from 2 to 8;
n is an integer of from 3 to 11, preferably from 3 to 7;
with the proviso that m+n is 5 to 19, preferably from 5 to 12;
   R¹ is —XNR⁶C(O)NR⁷R⁸; wherein
X is selected from —(CH₂)ₚ— and C₂₋₆alkenylene;
R⁶ and R⁸ are independently selected from hydrogen, C₁₋₆alkyl and C₃₋₇ cycloalkyl, wherein said C₁₋₆alkyl and C₃₋₇ cycloalkyl moieties may optionally be substituted by —CO₂H or —CO₂(C₁₋₄)alkyl;
R⁷ is selected from hydrogen, C₁₋₆alkyl, C₃₋₇cycloalkyl, —C(O)R⁹, phenyl, naphthyl, hetaryl, and phenyl(C₁₋₄alkyl)- and R⁷ is optionally substituted by 1 or 2 groups independently selected from halo, hydroxy, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H, —CO₂(C₁₋₄alkyl) and CONR¹⁰R¹¹;
R⁹ is selected from C₁₋₆alkyl, C₃₋₇cycloalkyl, —CO₂H, CO₂(C₁₋₄alkyl), phenyl, naphthyl, hetaryl, and phenyl(C₁₋₄alkyl)- and R⁹ is optionally substituted by 1 or 2 groups independently selected from halo, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H,
—CO₂(C₁₋₄alkyl);
R¹⁰ and R¹¹ each independently represent hydrogen,
C₁₋₄alkyl or C₃₋₇ cycloalkyl, and
p is an integer from 0 to 6, preferably from 0 to 4;
or R¹ is cyclised such that R⁸ forms a bond with the phenyl ring to which R¹ is attached, via the ring carbon atom adjacent to R¹, so as to form a moiety of the formula: 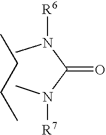
   R² is selected from hydrogen, C₁₋₆alkyl, C₁₋₆alkoxy, phenyl, halo, and C₁₋₆haloalkyl;
   R³ is selected from hydrogen, hydroxy, C₁₋₆alkyl, halo, C₁₋₆alkoxy, phenyl, C₁₋₆haloalkyl, and —SO₂NR¹²R¹³;
wherein R¹² and R¹³ are independently selected from hydrogen, C₁₋₆alkyl, C₃₋₆cycloalkyl, phenyl, and phenyl (C₁₋₄alkyl), or R¹² and R¹³, together with the nitrogen to which they are bonded, form a 5-, 6-, or 7-membered nitrogen containing ring;
and R¹² and R¹³ are each optionally substituted by one or two groups selected from halo, C₁₋₆alkyl, and C₁₋₆haloalkyl;
   R⁴ and R⁵ are independently selected from hydrogen and C₁₋₄alkyl with the proviso that the total number of carbon atoms in R⁴ and R⁵ is not more than 4;
   with the provisos that:
a) when R², R³, R⁴, R⁵, and R⁶ each denote hydrogen, m is 5, n is 2, and X denotes —(CH₂)ₚ— and is in the para position relative to the —O—(CH₂)ₙ— link, and p is 0, then R⁷ and R⁸ are not both hydrogen; and
b) when R², R³, R⁴, R⁵, and R⁶ each denote hydrogen, m is 5, n is 4, and X denotes —(CH₂)ₚ— and is in the para position relative to the —O—(CH₂)ₙ— link, and p is 0, then R⁷ and R⁸ are not both methyl.

Compounds of formula (I) wherein R⁶, R⁷ and R⁸ are each selected from hydrogen or C₁₋₄alkyl represent a selection from within GB2,159,191.

In the definition of R¹² and R¹³, the term “5-, 6-, or 7-membered nitrogen containing ring” means a 5-, 6-, or 7-membered saturated or unsaturated ring which includes a nitrogen atom and optionally 1 or 2 other heteroatoms independently selected from nitrogen, sulphur, and oxygen. Suitable examples of such a ring include piperidinyl, morpholinyl, and piperazinyl.

In the definition of R⁷, the term “hetaryl” means a 5- or 6-membered heteroaromatic ring, such as thienyl, pyrimidine, or pyridyl.

In the definition of X, the term alkenylene includes both cis and trans structures. Suitably examples of alkenylene groups include —CH═CH—.

In the compounds of formula (I) R¹ is preferably as defined hereinafter.

R² is preferably hydrogen.

R³ is preferably hydrogen, C₁₋₆haloalkyl or C₁₋₆alkyl.

In the compounds of formula (I), R⁴ and R⁵ are preferably independently selected from hydrogen and methyl, more preferably R⁴ and R⁵ are both hydrogen.

R⁶ and R⁸ preferably each independently represent hydrogen.

R⁷ is preferably selected from hydrogen, C₁₋₆alkyl; C₁₋₆alkyl substituted by a group selected from CO₂H, CO₂(C₁₋₄alkyl), CONH₂, and CONH(C₃₋₇cycloalkyl); phenyl; phenyl substituted by a group selected from halo, C₁₋₆alkyl, haloC₁₋₆alkyl and hydroxy; heteroaryl (eg. pyridyl or pyrimidinyl); C₃₋₇cycloalkyl; COPh and COCO₂H.

In the compounds of formula (I), m is suitably 3, 4 or 5, and preferably m is 5, and n is suitably 3 to 6 and preferably n is 3 or 4. More preferably n is 5 or 6 and n is 3 or 4 such that the sum of m+n is 8, 9 or 10, most preferably 9.

According to a preferred aspect of the invention, there is provided a compound of formula (Ia) 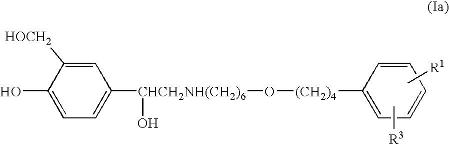
or a salt, solvate, or physiologically functional derivative thereof, wherein R¹ and R³ are as defined above for formula (I).

According to a further preferred aspect of the invention, there is provided a compound of formula (Ib) 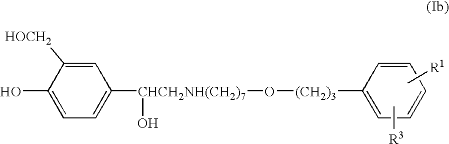
or a salt, solvate, or physiologically functional derivative thereof, wherein R¹ and R³ are as defined above for formula (I).

In the compounds of formulae (I), (Ia) and (Ib), the group R¹ is preferably attached to the meta-position relative to the —O—(CH₂)ₙ—, —O—(CH₂)₄— or —O—(CH₂)₃— link respectively.

In the compounds of formulae (I), (Ia) and (Ib), the group R¹ is preferably —(CH₂)ₚ—NHC(O)NHR⁷ and R⁷ is preferably hydrogen.

In the compounds of formulae (I), (Ia) and (Ib), p is most preferably 0, 1, or 2.

In the compounds of formulae (I), (Ia) and (Ib), R³ is preferably hydrogen, C₁₋₆haloalkyl, e.g. CF₃; or C₁₋₆alkyl, eg. methyl. The group R³ is suitably attached to the meta-position relative to the —O—(CH₂)ₙ—, —O—(CH₂)₄— or —O—(CH₂)₃— link respectively.

In a preferred embodiment when R⁶, R⁷ and R⁸ each represent hydrogen then at least one of R² or R³ represents a group other than hydrogen.

It is to be understood that the present invention covers all combinations of particular and preferred groups described hereinabove.

The compounds of formulae (I), (Ia) and (Ib) include an asymmetric centre, namely the carbon atom of the 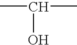
group. The present invention includes both (S) and (R) enantiomers either in substantially pure form or admixed in any proportions.

Similarly, where R⁴ and R⁵ are different groups, the carbon atom to which they are attached is an asymmetric centre and the present invention includes both (S) and (R) enantiomers at this centre either in substantially pure form or admixed in any proportions.

Thus the compounds of formulae (I), (Ia) and (Ib) include all enantiomers and diastereoisomers as well as mixtures thereof in any proportions.

Preferred compounds of the invention include:
N-(4-fluorophenyl)-N′-[3-(4-{[6-({(2R)-2-hydroxy-2-[4-hydroxy-3-(hydroxymethyl)phenyl]ethyl}amino)hexyl]oxy}butyl)phenyl]urea;
N-(2,6-dichlorophenyl)-N′-[3-(4-{[6-({(2R)-2-hydroxy-2-[4-hydroxy-3-(hydroxymethyl)phenyl]ethyl}amino)hexyl]oxy}butyl)phenyl]urea acetate;
N-[3-(4-{[6-({(2R)-2-hydroxy-2-[4-hydroxy-3-(hydroxymethyl)phenyl]ethyl}-amino)hexyl]oxy}butyl)phenyl]-N′-(4-methylphenyl)urea;
({[3-(4-{[6-({(2R)-2-hydroxy-2-[4-hydroxy-3-(hydroxymethyl)phenyl]ethyl}-amino)hexyl]oxy}butyl)anilino]carbonyl}amino)acetic acid;
N-[3-(4-{[6-({(2R)-2-hydroxy-2-[4-hydroxy-3-(hydroxymethyl)phenyl]ethyl}-amino)hexyl]oxy}butyl)phenyl]-N′-[3-(trifluoromethyl)phenyl]urea;
N-(2,6-dimethylphenyl)-N′-[3-(4-{[6-({(2R)-2-hydroxy-2-[4-hydroxy-3-(hydroxymethyl)phenyl]ethyl}amino)hexyl]oxy}butyl)phenyl]urea;
3-(4-{[6-({(2R)-2-hydroxy-2-[4-hydroxy-3-(hydroxymethyl)phenyl]ethyl}-amino)hexyl]oxy}butyl)phenyl)-N′-phenylurea;
N-Ethyl-N′-[3-(4-{[6-({(2R)-2-hydroxy-2-[4-hydroxy-3-(hydroxymethyl)phenyl]ethyl}amino)hexyl]oxy}butyl)phenyl)urea;
Ethyl({[3-(4-{[6-({(2R)-2-hydroxy-2-[4-hydroxy-3-(hydroxymethyl)phenyl]ethyl}-amino)hexyl]oxy}butyl)anilino]carbonyl}amino)acetate;
N-cyclohexyl-N′-(3-(4-{[6-({(2R)-2-hydroxy-2-[4-hydroxy-3-(hydroxymethyl)phenyl]ethyl}amino)hexyl]oxy}butyl)phenyl)urea;
N-[4-(4-{[6-({(2R)-2-hydroxy-2-[4-hydroxy-3-(hydroxymethyl)phenyl]ethyl}amino)hexyl]oxy}butyl)phenyl]-N′-phenylurea;
N-Ethyl-N′-[4-(4-{[6-({(2R)-2-hydroxy-2-[4-hydroxy-3-(hydroxymethyl)phenyl]ethyl}amino)hexyl]oxy}butyl)phenyl]urea;
N-[3-(4-{[6-({(2R)-2-hydroxy-2-[4-hydroxy-3-(hydroxymethyl)phenyl]ethyl}amino)hexyl]oxy}butyl)phenyl]-N′-pyridin-3-ylurea;
N-[3-(4-{[6-({(2R)-2-hydroxy-2-[4-hydroxy-3-(hydroxymethyl)phenyl]ethyl-amino)hexyl]oxy}butyl)phenyl]-N′-pyrimidin-4-ylurea;
N-[3,5-bis(trifluoromethyl)phenyl]-N′-[3-(4-{[6-({(2R)-2-hydroxy-2-[4-hydroxy-3-(hydroxymethyl)phenyl]ethyl}amino)hexyl]oxy}butyl)phenyl]urea;
N-cyclohexyl-N′-[3-(4-{([6-({(2R)-2-hydroxy-2-[4-hydroxy-3-(hydroxymethyl)phenyl]ethyl}amino)hexyl]oxy}butyl)benzyl]urea;
N-Ethyl-N′-[3-(4-{[6-({(2R)-2-hydroxy-2-[4-hydroxy-3-(hydroxymethyl)phenyl]ethyl}amino)hexyl]oxy}butyl)benzyl]urea;
N-[3-(4-{[6-({(2R)-2-hydroxy-2-[4-hydroxy-3-(hydroxymethyl)phenyl]ethyl}amino)hexyl]oxy}butyl)benzyl]urea;
N-(4-fluorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea;
N-(3-chlorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea;
N-benzyl-N′-[3-(4-{[6-({(2R)-2-hydroxy-2-[4-hydroxy-3-(hydroxymethyl)phenyl]ethyl}amino)hexyl]oxy}butyl)benzyl]urea;
N-({[2-(4-{[6-({(2R)-2-hydroxy-2-[4-hydroxy-3-(hydroxymethyl)phenyl]ethyl}-amino)hexyl]oxy}butyl)benzyl]amino}carbonyl)glycine;
N-{2-[3-(4-{[6-({(2R)-2-hydroxy-2-[4-hydroxy-3-(hydroxymethyl)phenyl]ethyl}amino)hexyl]oxy}butyl)phenyl]ethyl}-N′-phenylurea;
N-[3-(4-{[6-({(2R)-2-hydroxy-2-[4-hydroxy-3-(hydroxymethyl)phenyl]ethyl}amino)hexyl]oxy}butyl)phenyl]urea;
N-[3-(3-{[7-({(2R)-2-hydroxy-2-[4-hydroxy-3-(hydroxymethyl)phenyl]ethyl}amino)heptyl]oxy}propyl)phenyl]urea;
N-[3-(5-{([5-({(2R)-2-hydroxy-2-[4-hydroxy-3-(hydroxymethyl)phenyl]ethyl}amino)pentyl]oxy}pentyl)phenyl]urea;
N-[3-(5-{[6-({(2R)-2-hydroxy-2-[4-hydroxy-3-(hydroxymethyl)phenyl]ethyl}amino)hexyl]oxy}pentyl)phenyl]urea;
N-[3-(4-{[6-({(2R)-2-hydroxy-2-[4-hydroxy-3-(hydroxymethyl)phenyl]ethyl}amino)hexyl]oxy}butyl)-5-(trifluoromethyl)phenyl]urea;
N-[3-(4-{[6-({(2R)-2-hydroxy-2-[4-hydroxy-3-(hydroxymethyl)phenyl]ethyl}amino)hexyl]oxy}butyl)-5-methylphenyl]urea;
N-[3-(4-{[6-({(2S)-2-hydroxy-2-[4-hydroxy-3-(hydroxymethyl)phenyl]ethyl}amino)hexyl]oxy}butyl)-5-methylphenyl]urea;
5-(4-{[6-({(2R)-2-hydroxy-2-[4-hydroxy-3-(hydroxymethyl)phenyl]ethyl}amino)hexyl]oxy}butyl)-1,3-dihydro-2H-benzimidazol-2-one;
N-benzoyl-N′-[3-(4-{[6-({(2R)-2-hydroxy-2-[4-hydroxy-3-(hydroxymethyl)phenyl]ethyl}amino)hexyl]oxy}butyl)phenyl]urea;
N-[2-(4-{[6-({(2R)-2-hydroxy-2-[4-hydroxy-3-(hydroxymethyl)phenyl]ethyl}amino)hexyl]oxy}butyl)phenyl]-N′-phenylurea;
N-[3-(4-{[6-({(2R)-2-hydroxy-2-[4-hydroxy-3-(hydroxymethyl)phenyl]ethyl}amino)hexyl]oxy}butyl)phenyl]-N′-(3-hydroxyphenyl)urea;
[({[3-(4-{[6-({(2R)-2-hydroxy-2-[4-hydroxy-3-(hydroxymethyl)phenyl]ethyl}amino)hexyl]oxy}butyl)phenyl]amino}carbonyl)amino](oxo) acetic acid;
N²-({[3-(4-{[6-({(2R)-2-hydroxy-2-[4-hydroxy-3-(hydroxymethyl)phenyl]ethyl}amino)hexyl]oxy}butyl)phenyl]amino}carbonyl)glycinamide;

N¹-cyclopentyl-N²-({[3-(4-{[6-({(2R)-2-hydroxy-2-[4-hydroxy-3-(hydroxymethyl)phenyl]ethyl}amino)hexyl]oxy}butyl)phenyl]amino}carbonyl)glycinamide;
N-(aminocarbonyl)-N-[3-(4-{[6-({(2R)-2-hydroxy-2-[4-hydroxy-3-(hydroxymethyl)phenyl]ethyl}amino)hexyl]oxy}butyl)phenyl)-□-alanine;
   and salts, solvates, and physiologically functional derivatives thereof.

Particularly preferred compounds of the invention include:
N-[3-(4-{[6-({(2R)-2-hydroxy-2-[4-hydroxy-3-(hydroxymethyl)phenyl]ethyl}amino)hexyl]oxy}butyl)phenyl]urea;
3-(4-{[6-({(2R)-2-hydroxy-2-[4-hydroxy-3-(hydroxymethyl)phenyl]ethyl}-amino)hexyl]oxy}butyl)phenyl)-N′-phenylurea;
N-[3-(4-{[6-({(2S)-2-hydroxy-2-[4-hydroxy-3-(hydroxymethyl)phenyl]ethyl}amino)hexyl]oxy}butyl)phenyl]urea;
3-(4-{[6-({(2S)-2-hydroxy-2-[4-hydroxy-3-(hydroxymethyl)phenyl]ethyl}-amino)hexyl]oxy}butyl)phenyl)-N′-phenylurea;
N-[3-(4-{[6-({2-hydroxy-2-[4-hydroxy-3-(hydroxymethyl)phenyl]ethyl}amino)hexyl]oxy}butyl)phenyl]urea;
3-(4-{[6-({(2-hydroxy-2-[4-hydroxy-3-(hydroxymethyl)phenyl]ethyl}-amino)hexyl]oxy}butyl)phenyl)-N′-phenylurea;
N-[3-(4-{[6-({(2S)-2-hydroxy-2-[4-hydroxy-3-(hydroxymethyl)phenyl]ethyl}amino)hexyl]oxy}butyl)-5-methylphenyl]urea; and

N-[3-(4-{[6-({(2R)-2-hydroxy-2-[4-hydroxy-3-(hydroxymethyl)phenyl]ethyl}amino)hexyl]oxy}butyl)-5-methylphenyl]urea;

and salts, solvates and physiologically functional equivalents thereof.

Particularly preferred compounds of the invention further include
N-[3-(4-{[6-({(2R)-2-hydroxy-2-[4-hydroxy-3-(hydroxymethyl)phenyl]ethyl}amino)hexyl]oxy}butyl)-5-methylphenyl]urea;
   and salts and solvates thereof.

Salts and solvates of compounds of formulae (I), (Ia) and (Ib) which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formulae (I), (Ia) and (Ib) and their pharmaceutically acceptable salts, solvates, and physiologically functional derivatives.

By the term “physiologically functional derivative” is meant a chemical derivative of a compound of formula (I), (Ia) or (Ib) having the same physiological function as the free compound of formula (I), (Ia) or (Ib), for example, by being convertible in the body thereto. According to the present invention, examples of physiologically functional derivatives include esters.

Suitable salts according to the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulponic (for example p-toluenesulphonic, benzenesulphonic, naphthalenesulphonic or naphthalenedisulphonic), salicylic, glutaric, gluconic, tricarballylic, cinnamic, substituted cinnamic (for example, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexyl amine and N-methyl-D-glucamine.

Pharmaceutically acceptable esters of the compounds of formulae (I), (Ia) and (Ib) may have a hydroxyl group converted to a C₁₋₆alkyl, aryl, aryl C₁₋₆alkyl, or amino acid ester.

As mentioned above, the compounds of formulae (I), (Ia) and (Ib) are selective β₂-adrenoreceptor agonists as demonstrated using functional or reporter gene readout from cell lines transfected with human beta-adrenoreceptors as described below. Compounds according to the present invention also have the potential to combine long duration of effect with rapid onset of action. Furthermore, certain compounds (e.g. particularly preferred compounds indicated above) have demonstrated pharmacokinetic attributes that lead to improved lung retention and reduced oral absorption in animal models relative to existing long-acting β₂-agonist bronchodilators. As such, compounds of the invention may be suitable for once-daily administration.

Therefore, compounds of formulae (I), (Ia) and (Ib) and their pharmaceutically acceptable salts, solvates, and physiologically functional derivatives have use in the prophylaxis and treatment of clinical conditions for which a selective β₂-adrenoreceptor agonist is indicated. Such conditions include diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis).

Other conditions which may be treated include premature labour, depression, congestive heart failure, skin diseases (e.g. inflammatory, allergic, psoriatic, and proliferative skin diseases), conditions where lowering peptic acidity is desirable (e.g. peptic and gastric ulceration) and muscle wasting disease.

Accordingly, the present invention provides a method for the prophylaxis or treatment of a clinical condition in a mammal, such as a human, for which a selective β₂-adrenoreceptor agonist is indicated, which comprises administration of a therapeutically effective amount of a compound of formula (I), (Ia) or (Ib), or a pharmaceutically acceptable salt, solvate, or physiologically functional derivative thereof. In particular, the present invention provides such a method for the prophylaxis or treatment of a disease associated with reversible airways obstruction such as asthma, chronic obstructive pulmonary disease (COPD), respiratory tract infection or upper respiratory tract disease. In a further aspect the present invention provides such a method for the prophylaxis or treatment of a clinical condition selected from premature labour, depression, congestive heart failure, skin diseases (e.g. inflammatory, allergic, psoriatic, and proliferative skin diseases), conditions where lowering peptic acidity is desirable (e.g. peptic and gastric ulceration) or muscle wasting disease.

In the alternative, there is also provided a compound of formula (I), (Ia) or (Ib) or a pharmaceutically acceptable salt, solvate, or physiologically functional derivative thereof for use in medical therapy, particularly, for use in the prophylaxis or treatment of a clinical condition in a mammal, such as a human, for which a selective β₂-adrenoreceptor agonist is indicated. In particular, there is provided a compound of formula (I), (Ia) or (Ib) or a pharmaceutically acceptable salt, solvate, or physiologically functional derivative thereof for the prophylaxis or treatment of a disease associated with reversible airways obstruction such as asthma, chronic obstructive pulmonary disease (COPD), respiratory tract infection or upper respiratory tract disease. In a further aspect, there is provided a compound of formula (I), (Ia) or (Ib) or a pharmaceutically acceptable salt, solvate, or physiologically functional derivative thereof for the prophylaxis or treatment of a clinical condition selected from premature labour, depression, congestive heart failure, skin diseases (e.g. inflammatory, allergic, psoriatic, and proliferative skin diseases), conditions where lowering peptic acidity is desirable (e.g. peptic and gastric ulceration) or muscle wasting disease.

The present invention also provides the use of a compound of formula (I), (Ia) or (Ib), or a pharmaceutically acceptable salt, solvate, or physiologically functional derivative thereof in the manufacture of a medicament for the prophylaxis or treatment of a clinical condition for which a selective β₂-adrenoreceptor agonist is indicated, for example a disease associated with reversible airways obstruction such as asthma, chronic obstructive pulmonary disease (COPD), respiratory tract infection or upper respiratory tract disease. In a further aspect, there is provided a compound of formula (I), (Ia) or (Ib), or a pharmaceutically acceptable salt, solvate, or physiologically functional derivative thereof in the manufacture of a medicament for the prophylaxis or treatment of a clinical condition selected from premature labour, depression, congestive heart failure, skin diseases (e.g. inflammatory, allergic, psoriatic, and proliferative skin diseases), conditions where lowering peptic acidity is desirable (e.g. peptic and gastric ulceration) and muscle wasting disease.

The amount of a compound of formula (I), (Ia) or (Ib), or a pharmaceutically acceptable salt, solvate or physiologically functional derivative thereof which is required to achieve a therapeutic effect will, of course, vary with the particular compound, the route of administration, the subject under treatment, and the particular disorder or disease being treated. The compounds of the invention may be administered by inhalation at a dose of from 0.0005 mg to 10 mg, preferably 0.005 mg to 0.5 mg. The dose range for adult humans is generally from 0.0005 mg to 100 mg per day and preferably 0.01 mg to 1 mg per day.

While it is possible for the compound of formula (I), (Ia) or (Ib), or a pharmaceutically acceptable salt, solvate, or physiologically functional derivative thereof to be administered alone, it is preferable to present it as a pharmaceutical formulation.

Accordingly, the present invention further provides a pharmaceutical formulation comprising a compound of formula (I), (Ia) or (Ib) or a pharmaceutically acceptable salt, solvate, or physiologically functional derivative thereof, and a pharmaceutically acceptable carrier or excipient, and optionally one or more other therapeutic ingredients.

Hereinafter, the term “active ingredient” means a compound of formula (I), (Ia) or (Ib), or a pharmaceutically acceptable salt, solvate, or physiologically functional derivative thereof.

The formulations include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients. Packaging of the formulation may be suitable for unit dose or multi-dose delivery. In the case of multi-dose delivery, the formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurised formulations will generally be retained in a canister (eg an aluminium canister) closed with a valve (eg a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means eg by micronisation. The desired fraction may be separated out by air classification or sieving. Preferably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Intranasal sprays may be formulated with aqueous or non-aqueous vehicles with the addition of agents such as thickening agents, buffer salts or acid or alkali to adjust the pH, isotonicity adjusting agents or anti-oxidants.

Capsules and cartridges or for example gelatin, or blisters of for example laminated aluminium foil, for use in an inhaler or insulator may be formulated containing a powder mix of a compound of the invention and a suitable powder base such as lactose or starch.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose an acacia.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds and pharmaceutical formulations according to the invention may be used in combination with or include one or more other therapeutic agents, for example anti-inflammatory agents, anticholinergic agents (particularly an M₁, M₂, M₁/M₂ or M₃ receptor antagonist), other β₂-adrenoreceptor agonists, antiinfective agents (e.g. antibiotics, antivirals), or antihistamines. The invention thus provides, in a further aspect, a combination comprising a compound of formula (I) or a pharmaceutically acceptable salt, solvate or physiologically functional derivative thereof together with one or more other therapeutically active agents, for example, an anti-inflammatory agent (for example a corticosteroid or an NSAID), an anticholinergic agent, another β₂-adrenoreceptor agonist, an antiinfective agent (e.g. an antibiotic or an antiviral), or an antihistamine. Preferred are combinations comprising a compound of formula (I) or a pharmaceutically acceptable salt, solvate or physiologically functional derivative thereof together with a corticosteroid, and/or an anticholinergic, and/or a PDE-4 inhibitor. Preferred combinations are those comprising one or two other therapeutic agents.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

Suitable anti-inflammatory agents include corticosteroids and NSAIDs. Suitable corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126. Preferred corticosteroids include fluticasone propionate, and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, more preferably 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors of cytokine synthesis. Suitable other β₂-adrenoreceptor agonists include salmeterol (e.g. as the xinafoate), salbutamol (e.g. as the sulphate or the free base), formoterol (e.g. as the fumarate), fenoterol or terbutaline and salts thereof.

Of particular interest is use of the compound of formula (I) in combination with a phosphodiesterase 4 (PDE4) inhibitor or a mixed PDE3/PDE4 inhibitor. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family as well as PDE4. Generally it is preferred to use a PDE4 inhibitor which has an IC₅₀ ratio of about 0.1 or greater as regards the IC₅₀ for the PDE4 catalytic form which binds rolipram with a high affinity divided by the IC₅₀ for the form which binds rolipram with a low affinity. For the purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

A method for determining IC₅₀ ratios is set out in U.S. Pat. No. 5,998,428 which is incorporated herein in full by reference as though set out herein. See also PCT application WO 00/51599 for another description of said assay.

The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE4 which binds rolipram with a high affinity over the IC₅₀ value for inhibiting the PDE4 catalytic activity of a form which binds rolipram with a low affinity using 1 μM[³H]-cAMP as the substrate.

Examples of useful PDE4 inhibitors are:
(R)-(+)-1-(4-bromobenzyl)-4-[(3-cyclopentyloxy)-4-methoxyphenyl]-2-pyrrolidone;
(R)-(+)-1-(4-bromobenzyl)-4-[(3-cyclopentyloxy)-4-methoxyphenyl]-2-pyrrolidone;
3-(cyclopentyloxy-4-methoxyphenyl)-1-(4-N′-[N2-cyano-S-methyl-isothioureido]benzyl)-2-pyrrolidone;
cis 4-cyano-4-(3-cyclopentyloxy-4-methoxyphenyl)cyclohexan-1-carboxylic acid];
cis-[4-cyano-4-(3-cyclopropylmethoxy-4-difluoromethoxyphenyl)cyclohexan-1-ol];
(R)-(+)-ethyl[4-(3-cyclopentyloxy-4-methoxyphenyl)pyrrolidine-2-ylidene]acetate; and
(S)-(−)-ethyl[4-(3-cyclopentyloxy-4-methoxyphenyl)pyrrolidine-2-ylidene]acetate.

Most preferred are those PDE4 inhibitors which have an IC₅₀ ratio of greater than 0.5, and particularly those compounds having a ratio of greater than 1.0. Preferred compounds are cis 4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]; these are examples of compounds which bind preferentially to the low affinity binding site and which have an IC₅₀ ratio of 0.1 or greater.

Other compounds of interest include:

Compounds set out in U.S. Pat. No. 5,552,438 issued 3 Sep. 1996; this patent and the compounds it discloses are incorporated herein in full by reference. The compound of particular interest, which is disclosed in U.S. Pat. No. 5,552,438, is cis-4-cyano-4-[3-(cyclopentyloxy)-4-methoxyphenyl]cyclohexane-1-carboxylic acid (also known as cilomalast) and its salts, esters, pro-drugs or physical forms;

AWD-12-281 from elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P 2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Other possible PDE-4 and mixed PDE3/PDE4 inhibitors include those listed in WO01/13953, the disclosure of which is hereby incorporated by reference.

Suitable anticholinergic agents are those compounds that act as antagonists at the muscarinic receptor, in particular those compounds which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines. These drugs, particularly the salt forms, are readily available from a number of commercial sources or can be made or prepared from literature data via, to with:

Atropine—CAS-51-55-8 or CAS-51-48-1 (anhydrous form), atropine sulfate—CAS-5908-99-6; atropine oxide—CAS-4438-22-6 or its HCl salt—CAS-4574-60-1 and methylatropine nitrate—CAS-52-88-0.

Homatropine—CAS-87-00-3, hydrobromide salt—CAS-51-56-9, methylbromide salt—CAS-80-49-9.

Hyoscyamine (d, l)—CAS-101-31-5, hydrobromide salt—CAS-306-03-6 and sulfate salt—CAS-6835-16-1.

Scopolamine—CAS-51-34-3, hydrobromide salt—CAS-6533-68-2, methylbromide salt—CAS-155-41-9.

Preferred anticholinergics include ipratropium (e.g. as the bromide), sold under the name Atrovent, oxitropium (e.g. as the bromide) and tiotropium (e.g. as the bromide) (CAS-139404-48-1). Also of interest are: methantheline (CAS-53-46-3), propantheline bromide (CAS-50-34-9), anisotropine methyl bromide or Valpin 50 (CAS-80-50-2), clidinium bromide (Quarzan, CAS-3485-62-9), copyrrolate (Robinul), isopropamide iodide (CAS-71-81-8), mepenzolate bromide (U.S. Pat. No. 2,918,408), tridihexethyl chloride (Pathilone, CAS-4310-35-4), and hexocyclium methylsulfate (Tral, CAS-115-63-9). See also cyclopentolate hydrochloride (CAS-5870-29-1), tropicamide (CAS-1508-75-4), trihexyphenidyl hydrochloride (CAS-144-11-6), pirenzepine (CAS-29868-97-1), telenzepine (CAS-80880-90-9), AF-DX 116, or methoctramine, and the compounds disclosed in WO01/04118, the disclosure of which is hereby incorporated by reference.

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. The majority of these inhibitors, mostly first generation antagonists, have a core structure, which can be represented by the following formula:

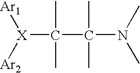

This generalized structure represents three types of antihistamines generally available: ethanolamines, ethylenediamines, and alkylamines. In addition, other first generation antihistamines include those which can be characterized as based on piperizine and phenothiazines. Second generation antagonists, which are non-sedating, have a similar structure-activity relationship in that they retain the core ethylene group (the alkylamines) or mimic the tertiary amine group with piperizine or piperidine. Exemplary antagonists are as follows:

Ethanolamines: carbinoxamine maleate, clemastine fumarate, diphenylhydramine hydrochloride, and dimenhydrinate.

Ethylenediamines: pyrilamine amleate, tripelennamine HCl, and tripelennamine citrate.

Alkylamines: chlropheniramine and its salts such as the maleate salt, and acrivastine.

Piperazines: hydroxyzine HCl, hydroxyzine pamoate, cyclizine HCl, cyclizine lactate, meclizine HCl, and cetirizine HCl.

Piperidines: Astemizole, levocabastine HCl, loratadine or its descarboethoxy analogue, and terfenadine and fexofenadine hydrochloride or another pharmaceutically acceptable salt.

Azelastine hydrochloride is yet another H₁ receptor antagonist which may be used in combination with a PDE4 inhibitor.

Examples of preferred anti-histamines include methapyrilene and loratadine.

The invention thus provides, in a further aspect, a combination comprising a compound of formula (I) a pharmaceutically acceptable salt, solvate or physiologically functional derivative thereof together with a PDE4 inhibitor.

The invention thus provides, in a further aspect, a combination comprising a compound of formula (I) a pharmaceutically acceptable salt, solvate or physiologically functional derivative thereof together with a corticosteroid.

The invention thus provides, in a further aspect, a combination comprising a compound of formula (I) a pharmaceutically acceptable salt, solvate or physiologically functional derivative thereof together with an anticholinergic.

The invention thus provides, in a further aspect, a combination comprising a compound of formula (I) a pharmaceutically acceptable salt, solvate or physiologically functional derivative thereof together with an antihistamine.

The invention thus provides, in a further aspect, a combination comprising a compound of formula (I) a pharmaceutically acceptable salt, solvate or physiologically functional derivative thereof together with a PDE4 inhibitor and a corticosteroid.

The invention thus provides, in a further aspect, a combination comprising a compound of formula (I) a pharmaceutically acceptable salt, solvate or physiologically functional derivative thereof together with an anticholinergic and a PDE-4 inhibitor.

The combinations referred to above may conveniently be presented for use in the form of a pharmaceutical formulation and thus pharmaceutical formulations comprising a combination as defined above together with a physiologically acceptable diluent or carrier represent a further aspect of the invention.

The individual compounds of such combinations may be administered either sequentially or simultaneously in separate or combined pharmaceutical formulations. Appropriate doses of known therapeutic agents will be readily appreciated by those skilled in the art.

According to a further aspect of the invention, there is provided a process for preparing a compound of formula (I), (Ia) or (Ib) or a salt, solvate, or physiologically functional derivative thereof which comprises a process (a) to (f) as defined below followed by the following steps in any order:
(i) optional removal of any protecting groups;
(ii) optional separation of an enantiomer from a mixture of enantiomers;
(iii) optional conversion of the product to a corresponding salt, solvate, or physiologically functional derivative thereof.

In one general process (a), a compound of formula (I), (Ia) or (Ib) may be obtained by deprotection of a protected intermediate, for example of formula (I): 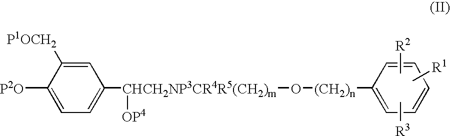
or a salt or solvate thereof, wherein R¹, R², R³, R⁴, R⁵, m, and n are as defined for the compound of formula (I), (Ia) or (Ib), and P¹, P², P³ and P⁴ are each independently either hydrogen or a protecting group provided that at least one of P¹, P², P³ and P⁴ is a protecting group.

Suitable protecting groups may be any conventional protecting group such as those described in “Protective Groups in Organic Synthesis” by Theodora W Greene and Peter G M Wuts, 3rd edition (John Wiley & Sons, 1999). Examples of suitable hydroxyl protecting groups represented by P¹, P² and P⁴ are esters such as acetate ester, aralkyl groups such as benzyl, diphenylmethyl, or triphenylmethyl, and tetrahydropyranyl. Examples of suitable amino protecting groups represented by P³ include benzyl, α-methylbenzyl, diphenylmethyl, triphenylmethyl, benzyloxycarbonyl, tert-butoxycarbonyl, and acyl groups such as trichloroacetyl or trifluoroacetyl.

As will be appreciated by the person skilled in the art, use of such protecting groups may include orthogonal protection of groups in the compounds of formula (II) to facilitate the selective removal of one group in the presence of another, thus enabling selective functionalisation of a single amino or hydroxyl function. For example, the —CH(OH) group may be orthogonally protected as —CHOP⁴ using, for example, a trialkylsilyl group such as triethylsilyl. A person skilled in the art will also appreciate other orthogonal protection strategies, available by conventional means as described in Theodora W Greene and Peter G M Wuts (see above).

The deprotection to yield a compound of formula (I), (Ia) or (Ib) may be effected using conventional techniques. Thus, for example, when P¹, P², and/or P³ is an aralkyl group, this may be cleaved by hydrogenolysis in the presence of a metal catalyst (e.g. palladium on charcoal).

When P¹ and/or P² is tetrahydropyranyl this may be cleaved by hydrolysis under acidic conditions. Acyl groups represented by P³ may be removed by hydrolysis, for example with a base such as sodium hydroxide, or a group such as trichloroethoxycarbonyl may be removed by reduction with, for example, zinc and acetic acid. Other deprotection methods may be found in Theodora W Greene and Peter G M Wuts (see above). In a particular embodiment of process (a), P¹ and P² may together represent a protecting group as in the compound of formula (III): 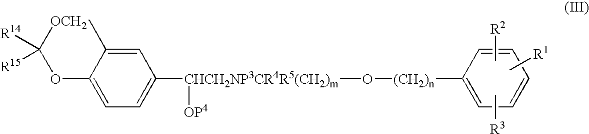
or a salt or solvate thereof, wherein R¹, R², R³, R⁴, R⁵, P³, P⁴, m, and n are as defined for the compound of formula (I), (Ia) or (Ib), and R¹⁴ and R¹⁵ are independently selected from hydrogen, C₁₋₆alkyl, or aryl or R¹⁴ and R¹⁵ together form a C₃₋₇cycloalkyl ring. In a preferred aspect, both R¹⁴ and R¹⁵ are methyl.

The compound of formula (III) may be converted to a compound of formula (I), (Ia) or (Ib) by hydrolysis with dilute aqueous acid, for example acetic acid or hydrochloric acid in a suitable solvent or by transketalisation in an alcohol, for example ethanol, in the presence of a catalyst such as an acid (for example, toluenesulphonic acid or a sulphonic acid ion exchange column such as SCX-2) or a salt (such as pyridinium tosylate) at normal or elevated temperature.

Compounds of formulae (II) and (III) wherein P³ is hydrogen may be prepared from the corresponding compound of formula (IV): 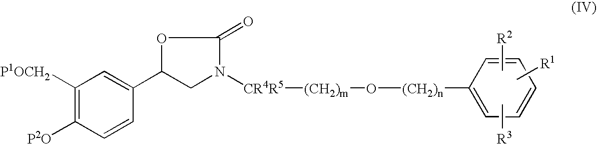
or a salt or solvate thereof, wherein R¹, R², R³, R⁴, R⁵, P¹, P² m, and n are as defined for the compound of formula (II) or (III).

The conversion of a compound of formula (IV) to a compound of formula (II) or (III) may be effected by treatment with a base, for example a non-aqueous base, such as potassium trimethylsilanolate, or an aqueous base such as aqueous sodium hydroxide, in a suitable solvent such as tetrahydrofuran.

Compounds of formula (IV) may be prepared from the corresponding compound of formula (V): 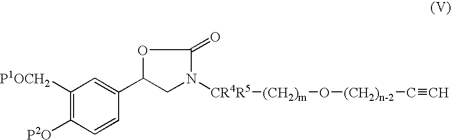
or a salt or solvate thereof, wherein R⁴, R⁵, P¹, P², m and n are as defined for the compound of formula (IV);
by coupling with a compound of formula (VI) or a precursor thereof: 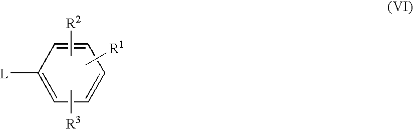
wherein R¹, R², and R³ are as defined for the compound of formula (IV) and L is a leaving group, such as a halo group (typically, bromo or iodo) or a sulphonate ester such as a haloalkyl sulphonate (typically, trifluoromethanesulphonate).

A suitable precursor of the compound of formula (VI) would be a compound of formula (VI) in which one or more of the substituents R¹, R², and R³ is a group which is convertible to the desired group R¹, R², and/or R³. For example, where R¹ is to be —(CH₂)ₚNR⁶C(O)NR⁷R⁸, a suitable precursor of the compound of formula (VI) would have the primary amine —(CH₂)ₚNH₂ in place of the substituent R¹, such that the desired substituent R¹ may be formed by reaction with the appropriate isocyanate (i.e. R⁷NCO) after the coupling with the compound of formula (V). Alternatively, R¹ is —XNCO (wherein X is as hereinbefore defined) which is coupled with an amine R⁷NH₂ using standard procedures.

The coupling of compound of formula (V) with a compound of formula (VI) or a precursor thereof is conveniently effected in the presence of a catalyst system such as bis(triphenylphosphine) palladium dichloride with an organic base such as a trialkylamine, for example, triethylamine, in a suitable solvent, for example acetonitrile or dimethylformamide. The resulting alkyne may then be reduced, either with or without being isolated to form the compound of formula (IV). The reduction may be effected by any suitable method such as hydrogenation in the presence of a catalyst, for example, palladium/charcoal or platinum oxide.

Alternatively, after coupling of a compound of formula (V) to a compound of formula (VI), the resulting compound may be treated with a base, for example a non-aqueous base such as potassium trimethylsilanolate, or an aqueous base such as aqueous sodium hydroxide, in a suitable solvent such as tetrahydrofuran, followed by reduction of the alkyne group to form a compound of formula (II) wherein P³ denotes hydrogen.

Compounds of formula (VI) are commercially available or may be prepared by methods well known to the person skilled in the art.

Compounds of formula (V) may be prepared by coupling a compound of formula (VII): 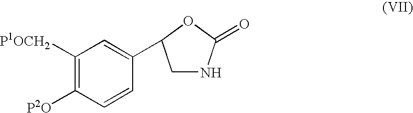
or a salt or solvate thereof, wherein P¹ and P² are as defined for the compound of formula (V) with a compound of formula (VIII):
L¹CR⁴R⁵(CH₂)ₘ—O—(CH₂)ₙ₋₂—C≡CH (VIII)
wherein R⁴, R⁵, m and n are as defined for the compound of formula (V) and L¹ is a leaving group, for example a halo group (typically bromo or iodo) or a sulphonate such as an alkyl sulphonate (typically, methanesulphonate), an arylsulphonate (typically, toluenesulphonate), or a haloalkyl sulphonate (typically, trifluoromethanesulphonate).

The coupling of a compound of formula (VII) with a compound of formula (VIII) may be effected in the presence of a base, such as a metal hydride, for example sodium hydride, or an inorganic base such as caesium carbonate, in an aprotic solvent, for example dimethylformamide.

Compounds of formula (VIII) may be prepared from the corresponding dihaloalkane and hydroxyalkyne by conventional chemistry, typically in the presence of an inorganic base, such as aqueous sodium hydroxide, under phase transfer conditions in the presence of a salt such as tetraalkylammonium bromide.

Compounds of formula (VII) may be prepared by ring closure of a compound of formula (IX): 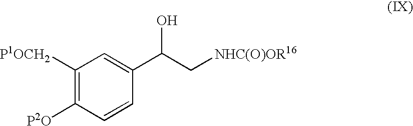
wherein P¹ and P² are as defined for the compound of formula (VII) and R¹⁵ is C₁₋₆alkyl, for example tert-butyl, or aryl, for example phenyl. The ring closure may be effected by treatment with a base, such as a metal hydride, for example sodium hydride, in the presence of an aprotic solvent, for example, dimethylformamide. Preparation of compounds (VII) is also described in WO02/066422.

Compounds of formula (IX) may be prepared from the corresponding ketone of formula (X): 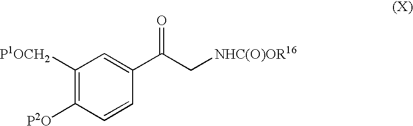
wherein P¹ and P² and R¹⁶ are as defined for the compound of formula (IX), by reduction by any suitable method, for example by treatment with borane, in the presence of a chiral catalyst, such as CBS-oxazaborolidine, in a suitable solvent such as tetrahydrofuran.

The compound of formula (X) may be prepared from the corresponding halide of formula (XI) 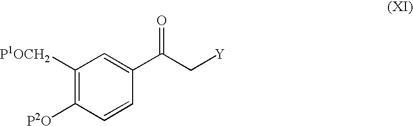
wherein P¹ and P² are as defined for the compound of formula (X) and Y is halo, suitably bromo.

The conversion of a compound of formula (XI) to a compound of formula (X) may be effected by reaction with the protected amine HN(COOR¹⁶)₂ wherein R¹⁵ is as defined for the compound of formula (X) in the presence of an inorganic base such as caesium carbonate, followed by selective removal of one of the COOR¹³ groups, for example by treatment with an acid such as trifluoroacetic acid.

Compounds of formula (XI) may be prepared from the corresponding compound having free hydroxymethyl and hydroxy substituents by forming the protected groups P¹OCH₂— and P²O wherein P¹ and P² are as defined for the compound of formula (XI). Such methods are described in DE 3513885 (Glaxo).

Compounds of formulae (II) or (III) wherein P³ is hydrogen or a protecting group may be prepared for example by analogous methods to those described in processes (b)-(f) below.

In a further process (b), a compound of formula (I), (Ia) or (Ib) or a compound of formula (II) or (III) may be obtained by alkylation of an amine of formula (XII) 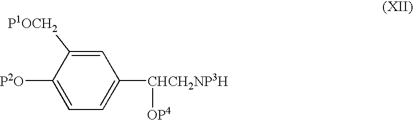
wherein P¹, P², P³ and P⁴ are each independently either hydrogen or a protecting group. Suitable protecting groups are discussed in the definition of compounds of formula (II);
with a compound of formula (XIII): 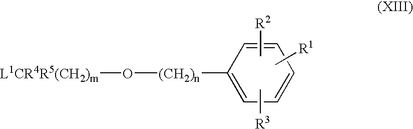
wherein R¹, R², R³, R⁴, R⁵, m, and n are as defined for the compound of formula (I), (Ia) or (Ib) and L¹ is a leaving group such as halo (typically bromo); followed by removal of any protecting groups present by conventional methods as described above for the deprotection of compounds of formula (II).

It will be appreciated that in this and subsequent processes a compound of formula (I) may be obtained directly where the groups P¹, P², P³ and P⁴ each represent hydrogen; alternatively when one or more of the groups P¹, P², P³ and P⁴ represents a protecting group, the product will be a compound of formula (II) or (III) which may then be deprotected according to process (a).

The reaction of compounds of formulae (XII) and (XIII) is optionally effected in the presence of an organic base such as a trialkylamine, for example, diisopropylethylamine, and in a suitable solvent for example dimethyl formamide.

Compounds of formula (XII) are known in the art (for example EP-A 0947498) or may be readily prepared by a person skilled in the art.

Compounds of formula (XIII) may be prepared by coupling a compound of formula (VI) as defined above, or a precursor thereof (wherein one or more of the substituents R¹, R² or R³ is a group which is convertible to the desired group R¹, R², or R³) with a compound of formula (VIII) as shown above wherein R⁴, R⁵, m, and n are as defined for the compound of formula (XIII) and L¹ is a leaving group as defined above.

Suitable precursors of the compounds of formula (VI) for this purpose may be designed using the same principles as described above in relation to the coupling of a compound of formula (VI) with a compound of formula (V).

The coupling of a compound of formula (VIII) with a compound (VI) may be effected by methods analogous to those described above for coupling a compound of formula (V) with a compound of formula (VI), followed by reduction of the resulting alkyne, also as described above. If necessary, the substituents R¹, R², and/or R³ may be formed by conventional conversions where a precursor is present.

Alternatively, a compound of formula (XIII) may be prepared by reacting an olefin of formula (XIV):
L¹CR⁴R⁵(CH₂)ₘ—O—(CH₂)ₙ₋₂CH═CH₂ (XIV)
wherein L¹, R⁴, R⁵, m and n are as defined for formula (VIII),
with a compound of formula (VI): 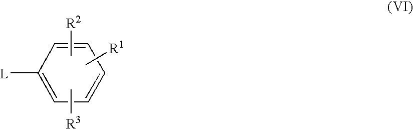
as hereinbefore defined.

The compound of formula (XIV) is initially reacted with 9-borabicyclo[3.3.1]nonane and followed by coupling with the compound (VI) in the presence of a catalyst such as palladium acetate and triphenylphosphine and a base such as aqueous potassium phosphate.

Compounds of formula (XIV) may be prepared by standard methods well known to those skilled in the art, for example in similar manner to the preparation of compounds of formula (VIII) described hereinabove.

In a yet further process (c) a compound of formula (I), (Ia), (Ib), (II) or (III) may be obtained by reduction of a compound of formula (XV): 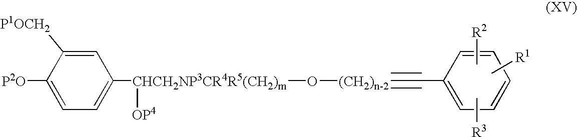
Wherein R¹, R², R³, R⁴, R⁵, m and n are as defined for formula (I) and P¹, P², P³ and P⁴ are each independently hydrogen or a protecting group as defined above.

The reduction may be effected by any suitable method such as hydrogenation in the presence of a catalyst, for example, palladium/charcoal or platinum oxide.

It will be appreciated that where P¹, P², P³ and P⁴ each represent hydrogen, the reduction will yield a compound of formula (I), but where one or more of P¹, P², P³ and P⁴ represent a protecting group then reduction will yield a compound of formula (II) or (III), which may then be deprotected to give a compound of formula (I).

A compound of formula (XV) may be prepared by reacting a compound of formula (XII) as herein before defined with a compound of formula (XVI): 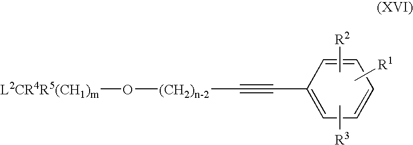
wherein R¹, R², R³, R⁴, R⁵, m, and n are as defined for the compound of formula (I), (Ia) or (Ib) and L² is as defined for L and L¹ above.

The reaction of compounds of formulae (XV) and (XVI) is optionally effected in the presence of an organic base such as a trialkylamine, for example, diisopropylethylamine, and in a suitable solvent for example N,N-dimethylformamide.

The compound of formula (XVI) may be prepared by coupling a compound of formula (VI) as defined above with a compound of formula (VIII) as defined above, as described for the first stage of the preparation of compounds (XIII), without the reduction step.

An alkyne of formula (XVI) may also be prepared by reacting a compound of formula (XVII):
L²CR⁴R⁵(CH₂)ₘL³ (XVII)
Wherein R⁴, R⁵ and m are as defined hereinabove and L² and L³ each represent a leaving group, which groups may independently be selected for example from those defined above for L and L¹, with a compound of formula (XVIII): 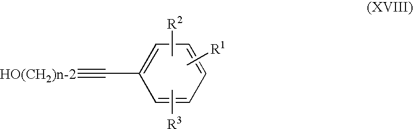
using conventional methods, for example as described for the preparation of compounds (VIII).

Compounds of formula (XVIII) may be prepared by reacting a hydroxyalkyne
HO(CH₂)n-2≡
with a compound of formula (VI) using methods analogous to those described above for coupling a compound (V) with a compound (VI).

In a further process (d) a compound of formula (I), (Ia), (Ib) (II) or (III) may be prepared by reacting a compound of formula (XIX): 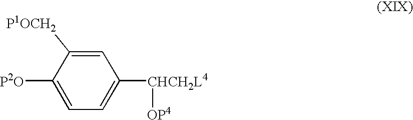
P¹, P² and P⁴ are as hereinbefore defined and L⁴ is a leaving group as defined above for groups L-L³ with an amine of formula (XX): 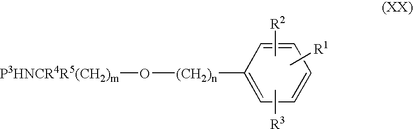
followed by removal of any protecting groups present by conventional methods as described above for the deprotection of compounds of formula (II).

The reaction may be effected using conventional conditions for such displacement reactions.

Compounds of formula (XIX) may be prepared by methods known in the art.

Compounds of formula (XX) may be prepared by reacting a compound of formula (XIII) with an amine P³NH₂.

In a further process (e) a compound of formula (I), (Ia), (Ib), (II) or (III) may be prepared by removal of a chiral auxiliary from a compound of formula (Ia): 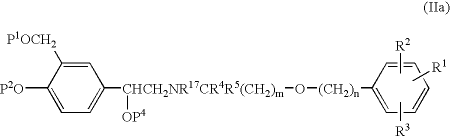
wherein R¹-R⁵, m and n are as defined for formula (I), P¹, P² and P⁴ each independently represent hydrogen or a protecting group and R¹⁷ represents a chiral auxiliary.

A “chiral auxiliary” is a moiety that is introduced into a molecule to influence the stereochemistry of the product formed, and is removed in whole or part at a later time. A chiral auxiliary may simultaneously function as a protecting group.

Many chiral auxiliaries are commercially available, and persons skilled in the art would choose one based on the properties desired i.e. the absolute stereochemistry desired and compatibility with the processes being used. Chiral auxiliaries suitable for use in this process include but are not limited to the S-isomer and/or the R-isomer of phenyl glycinol and substituted derivatives thereof.

The chiral auxiliary is preferably a moiety of the formula: 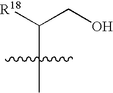
or a single enantiomer thereof, wherein R¹⁸ represents C₁₋₆alkyl or optionally substituted phenyl or benzyl wherein the optional substitution is one or more independently selected from C₁₋₆alkyl, halogen, hydroxy, C₁₋₆alkoxy or nitro e.g. para-hydroxyphenyl.

More preferably the chiral auxiliary is a moiety: 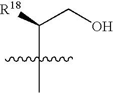
wherein R¹⁸ is as defined above. Alternatively it may be a moiety of formula: 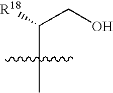
wherein R¹⁸ is as defined above.

Preferably R¹ represents phenyl optionally substituted as described above. Most preferably R¹⁸ represents unsubstituted phenyl.

The chiral auxiliary in this process may typically be removed by hydrogenolysis using for example a palladium on carbon catalyst or preferably using palladium hydroxide (Pearlman's catalyst). Advantageously when Pearlman's catalyst is used the removal of the chiral auxiliary is most efficient. This method of removal is especially suitable where R¹⁸ is phenyl or a substituted phenyl. Alternatively the nitrogen, to which the auxiliary is attached, may be derivatised under oxidising conditions to form the N-oxide before elimination by heating to give a secondary amine.

A compound of formula (IIa) may be prepared by reduction of the corresponding alkyne of formula (XVa): 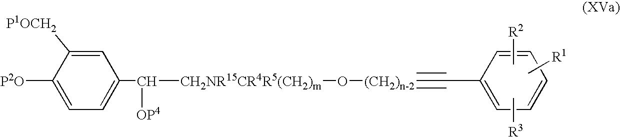
wherein R¹, R², R³, R⁴, R⁵, m and n are as defined for formula (I) and P¹, P², P⁴ and R¹⁷ are as defined for formula (IIa).

Preferably in the compounds of formulae (IIa) and (XVa) the protecting groups P¹ and P² together form a group —CR¹⁴R¹⁵— as in the compounds of formula (III).

Reduction of an alkyne of formula (XVa) may be effected by methods well known in the art, for example by catalytic hydrogenation, using palladium on charcoal or more preferably palladium hydroxide (Pearlman's catalyst). The chiral auxiliary may also be removed under reductive conditions. Advantageously, therefore the reduction of the alkyne and removal of the chiral auxiliary may be effected concomitantly in a ‘one-pot’ reaction.

An alkyne of formula (XVa) may be prepared by reaction of a compound of formula (XXI): 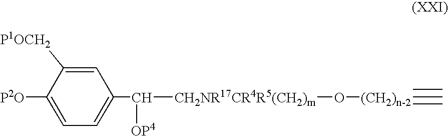
wherein R⁴, R⁵, m and n are as defined for formula (I) and P¹, P², P⁴ and R¹⁷ are as defined for formula (IIa) with a compound of formula (VI) under conditions described above for coupling of compounds (V) and (VI).

A compound of formula (XXI) may be prepared by reacting a compound of formula (XIIa): 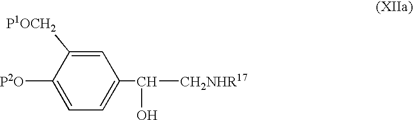
with an aldehyde of formula (XXII): 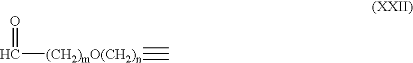
using known methods for effecting reductive amination, e.g. sodium triacetoxyborohydride in a solvent such as chloroform.

An aldehyde of formula (XXII) may be prepared from a corresponding halide of formula (VIII) using standard techniques such as treatment with sodium bicarbonate in a solvent such as DMSO at elevated temperature, preferably in the range 130-160° C.

A compound of formula (XIIa) may be prepared from a compound of formula (Xa): 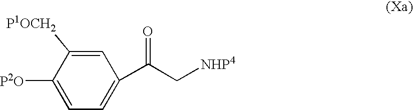
wherein P¹, P² and P⁴ are as defined for formula (IIa), by treatment with a reducing agent such as a hydride source e.g. sodium borohydride. Preferably this process takes place in the presence of an inert metal salt such as calcium chloride suitably at non-extreme temperatures e.g. below ambient, such as 0° C. This allows the desired stereochemistry to be introduced efficiently with good enantiomeric excess at an early stage in the synthesis, using inexpensive and relatively harmless reagents. Furthermore, the enantiomeric excess may be increased by recrystallisation of the product of this process.

A compound of formula (Xa) may be prepared from a compound of formula (XI) as hereinbefore defined by reaction with an appropriate chiral amine, e.g. (S)-phenylglycinol, in the presence of a non-nucleophilic base in an inert solvent at non-extreme temperatures.

A detailed description of a process analogous to Route (e) may be found in published International Application Number WO/0196278.

In the above process (e) it is preferred that the protecting groups P¹ and P² together form a cyclic protecting group as depicted in formula (III).

According to a further process (f) a compound of formula (I), (Ia), (Ib), (II) or (III) wherein R¹ is —XNR⁶C(O)NR⁷R⁸, X is a bond, R⁶ is hydrogen and R⁷ is —CH₂CONR¹⁰R¹¹, may be prepared by reacting a compound (XXIII): 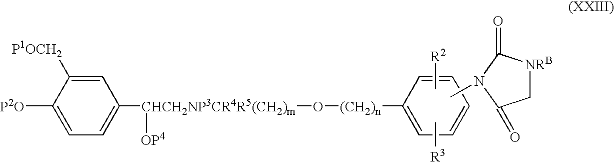
wherein P¹, P², P³, P⁴, R², R³, R⁴, R⁵ and R⁸ are as defined above,
with an amine of formula HNR¹⁰R¹¹,
wherein R¹⁰ and R¹¹ are as hereinbefore defined. The reaction is conveniently effected in a solvent such as an alcohol, eg. methanol or ethanol.

Compounds of formula (XXXIII) are known from WO02/070490.

It will be appreciated that in any of the routes (a) to (f) described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly.

The enantiomeric compounds of the invention may be obtained (i) by separation of the components of the corresponding racemic mixture, for example, by means of a chiral chromatography column, enzymic resolution methods, or preparing and separating suitable diastereoisomers, or (ii) by direct synthesis from the appropriate chiral intermediates by the methods described above.

Optional conversions of a compound of formula (I), (Ia) or (Ib) to a corresponding salt may conveniently be effected by reaction with the appropriate acid or base. Optional conversion of a compound of formula (I), (Ia) or (Ib) to a corresponding solvate or physiologically functional derivative may be effected by methods known to those skilled in the art.

According to a further aspect, the present invention provides novel intermediates for the preparation of compounds of formula (I), (Ia) or (Ib), for example:

compounds of formula (II) and (III) as defined above, or an optical isomer, a salt, or a protected derivative thereof; particularly, a compound selected from:

N-(3-{4-[(6-{[(2R)-2-(2,2-dimethyl-4H-1,3-benzodioxin-6-yl)-2-hydroxyethyl]amino}hexyl)oxy]butyl}phenyl)-N′-(4-fluorophenyl)urea
N-(3-{4-[(6-{[(2R)-2-(2,2-dimethyl-4H-1,3-benzodioxin-6-yl)-2-hydroxyethyl]amino}hexyl)oxy]butyl}phenyl)-N′-(2,6-dichlorophenyl)urea;
N-(3-{4-[(6-{[(2R)-2-(2,2-dimethyl-4H-1,3-benzodioxin-6-yl)-2-hydroxyethyl]amino}hexyl)oxy]butyl}phenyl)-N′-(4-methylphenyl)urea
{[(3-{4-[(6-{[(2R)-2-(2,2-dimethyl-4H-1,3-benzodioxin-6-yl)-2-hydroxyethyl]amino}hexyl)oxy]butyl}anilino)carbonyl]amino}acetic acid
N-(3-{4-[(6-{[(2R)-2-(2,2-dimethyl-4H-1,3-benzodioxin-6-yl)-2-hydroxyethyl]amino}hexyl)oxy]butyl}phenyl)-N′-[3-(trifluoromethyl)phenyl]urea;
N-(3-{4-[(6-{[(2R)-2-(2,2-dimethyl-4H-1,3-benzodioxin-6-yl)-2-hydroxyethyl]amino}hexyl)oxy]butyl}phenyl)-N′-(2,6-dimethylphenyl)urea;
N-(3-{4-[(6-{[(2R)-2-(2,2-dimethyl-4H-1,3-benzodioxin-6-yl)-2-hydroxyethyl]amino}hexyl)oxy]butyl}phenyl)-N′-phenylurea
N-(3-{4-[(6-{[(2R)-2-(2,2-dimethyl-4H-1,3-benzodioxin-6-yl)-2-hydroxyethyl]amino}hexyl)oxy]butyl}phenyl)-N′-ethyl urea;
Ethyl {[(3-{4-[(6-{[(2R)-2-(2,2-dimethyl-4H-1,3-benzodioxin-6-yl)-2-hydroxyethyl]amino}hexyl)oxy]butyl}anilino)carbonyl]amino}acetate;
N-Cyclohexyl-N′-(3-{4-[(6-{[(2R)-2-(2,2-dimethyl-4H-1,3-benzodioxin-6-yl)-2-hydroxyethyl]amino}hexyl)oxy]butyl}phenyl)urea;
N-(4-{4-[(6-{[(2R)-2-(2,2-dimethyl-4H-1,3-benzodioxin-6-yl)-2-hydroxyethyl]amino}hexyl)oxy]butyl}phenyl)-N′-phenylurea;
N-(4-{4-[(6-{[(2R)-2-(2,2-dimethyl-4H-1,3-benzodioxin-6-yl)-2-hydroxyethyl]amino}hexyl)oxy]butyl}phenyl)-N′-ethylurea;
N-(3-{4-[(6-{[(2R)-2-(2,2-dimethyl-4H-1,3-benzodioxin-6-yl)-2-hydroxyethyl]amino}hexyl)oxy]butyl}phenyl)-N′-pyridin-3-ylurea;
N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}-N′-pyrimidin-4-ylurea;
N-[3,5-Bis(trifluoromethyl)phenyl]-N′-(3-{4-[(6-{[(2R)-2-(2,2-dimethyl-4H-1,3-benzodioxin-6-yl)-2-hydroxyethyl]amino}hexyl)oxy]butyl}phenyl)urea;
N-Cyclohexyl-N′-(3-{4-[(6-{[(2R)-2-(2,2-dimethyl-4H-1,3-benzodioxin-6-yl)-2-hydroxyethyl]amino}hexyl)oxy]butyl}benzyl)urea;
N-(3-{4-[(6-{[(2R)-2-(2,2-dimethyl-4H-1,3-benzodioxin-6-yl)-2-hydroxyethyl]amino}hexyl)oxy]butyl}benzyl)-N′-ethylurea;
N-(3-{4-[(6-{[(2R)-2-(2,2-dimethyl-4H-1,3-benzodioxin-6-yl)-2-hydroxyethyl]amino}hexyl)oxy]butyl}benzyl)urea;
N-(3-{4-[(6-{[(2R)-2-(2,2-dimethyl-4H-1,3-benzodioxin-6-yl)-2-hydroxyethyl]amino}hexyl)oxy]butyl}benzyl)-N′-(4-fluorophenyl)urea;
N-(3-Chlorophenyl)-N′-(3-{4-[(6-{[(2R)-2-(2,2-dimethyl-4H-1,3-benzodioxin-6-yl)-2-hydroxyethyl]amino}hexyl)oxy]butyl}benzyl)urea;
N-Benzyl-N′-(3-{4-[(6-{[(2R)-2-(2,2-dimethyl-4H-1,3-benzodioxin-6-yl)-2-hydroxyethyl]amino}hexyl)oxy]butyl}benzyl)urea;
N-{[(2-{4-[(6-{[(2R)-2-(2,2-dimethyl-4H-1,3-benzodioxin-6-yl)-2-hydroxyethyl]amino}hexyl)oxy]butyl}benzyl)amino]carbonyl}glycine;
N-[2-(3-{4-[(6-{[(2R)-2-(2,2-dimethyl-4H-1,3-benzodioxin-6-yl)-2-hydroxyethyl]amino}hexyl)oxy]butyl}phenyl)ethyl]-N′-phenylurea;
N-(3-{4-[(6-{[(2R)-2-(2,2-dimethyl-4H-1,3-benzodioxin-6-yl)-2-hydroxyethyl]amino}hexyl)oxy]butyl}phenyl)urea;
N-(3-{3-[(7-{[(2R)-2-(2,2-dimethyl-4H-1,3-benzodioxin-6-yl)-2-hydroxyethyl]amino}heptyl)oxy]propyl}phenyl)urea;
N-(3-{5-[(5-{[(2R)-2-(2,2-dimethyl-4H-1,3-benzodioxin-6-yl)-2-hydroxyethyl]amino}pentyl)oxy]pentyl}phenyl)urea;
N-(3-{5-[(6-{[(2R)-2-(2,2-dimethyl-4H-1,3-benzodioxin-6-yl)-2-hydroxyethyl]amino}hexyl)oxy]pentyl}phenyl)urea;
N-[3-{4-[(6-{[(2R)-2-(2,2-dimethyl-4H-1,3-benzodioxin-6-yl)-2-hydroxyethyl]amino}hexyl)oxy]butyl}-5-(trifluoromethyl)phenyl]urea;
N-(3-{4-[(6-{[(2R)-2-(2,2-dimethyl-4H-1,3-benzodioxin-6-yl)-2-hydroxyethyl]amino}hexyl)oxy]butyl}-5-methylphenyl)urea;
5-[4-({6-[(5R)-5-(2,2-dimethyl-4H-1,3-benzodioxin-6-yl)-2-oxo-1,3-oxazolidin-3-yl]hexyl}oxy)butyl]-1,3-dihydro-2H-benzimidazol-2-one;
5-(4-{[6-({(2R)-2-hydroxy-2-[4-hydroxy-3-(hydroxymethyl)phenyl]ethyl}amino)hexyl]oxy}butyl)-1,3-dihydro-2H-benzimidazol-2-one;
N-(2-{4-[(6-{[(2R)-2-(2,2-dimethyl-4H-1,3-benzodioxin-6-yl)-2-hydroxyethyl]amino}hexyl)oxy]butyl}phenyl)-N′-phenylurea;
N-{3-[4-({6-[(5R)-5-(2,2-dimethyl-4H-1,3-benzodioxin-6-yl)-2-oxo-1,3-oxazolidin-3-yl]hexyl}oxy)butyl]phenyl}-N′-(3-hydroxyphenyl)urea; and
({[(3-{4-[(6-{[(2R)-2-(2,2-dimethyl-4H-1,3-benzodioxin-6-yl)-2-hydroxyethyl]amino}hexyl)oxy]butyl}phenyl)amino]carbonyl}amino)(oxo)acetic acid.

For a better understanding of the invention, the following Examples are given by way of illustration.

### SYNTHETIC EXAMPLES

Throughout the examples, the following abbreviations are used:

LCMS: Liquid Chromatography Mass Spectrometry

MS mass spectrum

TSP+ve thermospray mass spectrum positive mode

RT: retention time

THF: tetrahydrofuran

DMF: N,N-dimethylformamide

EtOAc: ethyl acetate

EtOH: ethanol

MeOH: methanol

BBN: 9-borabicyclo[3.3.1]nonane

bp: boiling point

ca: circa

h: hour(s)

min: minute(s)

All temperatures are given in degrees centigrade.

Silica gel refers to Merck silica gel 60 Art number 7734.

Flash silica gel refers to Merck silica gel 60 Art number 9385.

Biotage refers to prepacked silica gel cartridges containing KP-Sil run on flash 121 chromatography module.

Bond Elut are prepacked cartridges used in parallel purifications, normally under vacuum. These are commercially available from Varian.

SCX-2 is a solid phase extraction column pre-packed with benzene sulfonic acid resin available from International Sorbent Technology.

LCMS was conducted on a Supelcosil LCABZ+PLUS column (3.3 cm×4.6 mm ID) eluting with 0.1% HCO₂H and 0.01 M ammonium acetate in water (solvent A), and 0.05% HCO₂H 5% water in acetonitrile (solvent B), using the following elution gradient 0-0.7 min 0% B, 0.7-4.2 min 100% B, 4.2-5.3 min 100% B, 5.3-5.5 min 0% B at a flow rate of 3 ml/min. The mass spectra were recorded on a Fisons VG Platform spectrometer using electrospray positive and negative mode (ES+ve and ES−ve).

HPLC System for Examples 39-42:

| | |
|---|---|
| Column | Phenomenex Luna C18(2), 50 × 2.0 mm |
| Mobile phase | A = water containing 0.5% trifluoroacetic acid |
| | B = acetonitrile containing 0.05% trifluoroacetic acid |
| Gradient | 0% to 95% B over 8 minutes |
| Flow rate | 1 ml/min |
| Temperature | 40° C. |
| Detection | UV at 220 nm |

GC System for Examples 39-42

| | |
|---|---|
| Column | HP-5, 30 m × 0.32 mm × 0.32 μm |
| Column flow | Helium @ ˜2 ml/min |
| Injector temp | 260° C. |
| Detector temp | 280° C. |
| Oven programme | 40° C. for 3 mins |
| | heat to 240° C. at 20° C./min |
| | hold for 2 mins |

### Example 1

### N-(4-Fluorophenyl)-N′-[3-(4-{[6-({(2R)-2-hydroxy-2-[4-hydroxy-3-(hydroxymethyl)phenyl]ethyl}amino)hexyl]oxy}butyl)phenyl]urea acetate

### i) Di(tert-butyl)2-(2,2-dimethyl-4H-1,3-benzodioxin-6-yl)-2-oxoethylimidodicarbonate

Caesium carbonate (70.4 g) was added to a stirred suspension of 2-bromo-1-(2,2-dimethyl-4H-1,3-benzodioxin-6-yl)ethanone, (Glaxo, DE 3513885, 1985) (61.8 g) and di-t-butyl iminodicarboxylate (47.15 g) in acetonitrile (600 ml) under nitrogen. After vigorous stirring at 21° for 24 h the mixture was diluted with water (ca 800 ml) and the product was extracted with diethyl ether (1 litre, then 200 ml). The combined organic layers were washed with brine, dried (MgSO₄) and concentrated to ca 400 ml. The white crystals were collected by filtration, washed with diethyl ether and dried to give the title compound (24.4 g) δ (CDCl₃) 7.78 (1H, dd, J 8, 2 Hz), 7.65 (1H, brs), 6.87 (1H, d, J 8 Hz), 4.97 (2H, s), 4.88 (2H, s), 1.56 (6H, s) and 1.48 (18H, s). Further concentration of the mother liquors gave additional product (13.8 g). A third crop (7.1 g) was obtained by chromatographing the mother liquors on silica gel, evaporating the appropriate eluate and triturating with diethyl ether.

### ii) tert-Butyl 2-(2,2-dimethyl-4H-1,3-benzodioxin-6-yl)-2-oxoethylcarbamate

Trifluoroacetic acid (92 ml) was added to a stirred solution of di(tert-butyl)2-(2,2-dimethyl-4H-1,3-benzodioxin-6-yl)-2-oxoethylimidodicarbonate, (352.55 g) in CH₂Cl₂ (3.6 litres) at 21° and the reaction was stirred for 1.5 h. Aqueous NaOH solution (1.75 litres) was added and after 10 min the phases were separated. The organic layer was washed with water, dried (MgSO₄) and evaporated to an oil. This was stored under high vacuum overnight and then triturated with hexane:ether (3:1) to give the crude product (226.61 g). This was purified by recrystallisation from diethyl ether to give the title compound (122.78 g). Further product (61.5 g) was obtained from the mother liquors by evaporation and chromatography on a Biotage using 15% ethyl acetate in hexane. LCMS RT=3.37 min.

### iii) tert-Butyl (2R)-2-(2,2-dimethyl-4H-1,3-benzodioxin-6-yl)-2-hydroxyethylcarbamate

A 2M solution of borane-dimethyl sulphide in THF (28 ml) was added slowly to a 1M solution of (R)-tetrahydro-1-methyl-3,3-diphenyl-1H,3H-pyrrolo[1,2-c][1,3,2]oxazaborole in toluene (56 ml) at 0° under nitrogen. A solution of tert-butyl 2-(2,2-dimethyl-4H-1,3-benzodioxin-6-yl)-2-oxoethylcarbamate, (108.2 g) in THF (1.3 litres) was added slowly keeping the temperature below 5° followed by 2M solution of borane-dimethyl sulphide in THF (252 ml) over 50 min. After 1 h, 2M HCl (170 ml) was added with cooling and the mixture was partitioned between EtOAc and water. The organic layer washed with saturated

NaHCO₃ solution and brine and dried (MgSO₄). The solution was concentrated and the product purified by chromatography on flash silica gel (800 g), eluting successively with hexane:EtOAc (4:1 then 3:1) to give the title compound (93.3 g). LCMS RT=3.31 min.

### iv) (5R)-5-(2,2-Dimethyl-4H-1,3-benzodioxin-6-yl)-1,3-oxazolidin-2-one

tert-Butyl (2R)-2-(2,2-dimethyl-4H-1,3-benzodioxin-6-yl)-2-hydroxyethylcarbamate, (86.37 g) in DMF (600 ml) was added dropwise to a stirred suspension of sodium hydride (60% oil dispersion, 11.9 g) in DMF (160 ml) with cooling such that the internal temperature remained at 0° under nitrogen. The mixture was stirred at 21° for 2 h. The mixture was recooled to 0° and 2M HCl (134 ml) was added. The mixture was diluted with water and the product was extracted with EtOAc twice. The solution washed with brine twice, dried (MgSO₄) and evaporated to give the title compound (63.55 g). LCMS RT=2.66 min.

### v) 6-Bromohexyl but-3-ynyl ether

3-Butyn-1-ol (42.4 ml) was stirred vigorously with 1,6-dibromohexane (260 ml) and tetrabutylammonium bisulphate (2.4 g) in 50% aqueous sodium hydroxide solution (200 ml) under nitrogen for 3 days. Water (ca 700 ml) was added and the organic layer was separated. The aqueous layer was extracted twice with CH₂Cl₂ (2×100 ml) and the combined organic layers were washed with water, dried (MgSO₄) and concentrated. The residue in petroleum ether (bp 40-600) was loaded onto a column of silica gel (1.5 kg) and the column was eluted with petroleum ether (bp 40-60°), then 10% diethyl ether in petroleum ether (bp 40-600) to give the title compound (103.3 g), δ (CDCl₃) 3.56 (2H, t, J 7 Hz), 3.47 (2H, t, J 7 Hz), 3.42 (2H, t, J 7 Hz), 2.45 (2H, m), 1.99 (1H, t, J 2 Hz), 1.87 (2H, m), 1.60 (2H, m) and 1.50-1.33 (4H, m).

### vi) (5R)-3-[6-(But-3-ynyloxy)hexyl]-5-(2,2-dimethyl-4H-1,3-benzodioxin-6-yl)-1,3-oxazolidin-2-one

(5R)-5-(2,2-Dimethyl-4H-1,3-benzodioxin-6-yl)-1,3-oxazolidin-2-one (10 g) in DMF (100 ml) was added dropwise to a stirred suspension of sodium hydride (60% oil dispersion, 2.33 g) in DMF (50 ml) with stirring under nitrogen and maintaining the internal temperature at 0°. Stirring was continued at 0-5° for 1 h. The mixture was recooled to 0° and a solution of 6-bromohexyl but-3-ynyl ether (14.7 g) in DMF (50 ml) was added over 1 min. The mixture was then stirred at 20-30° for 2 h. 2M HCl (9 ml) was added and the mixture was partitioned between water and diethyl ether. The aqueous layer was extracted with more diethyl ether and the combined organic layers were washed twice with brine. After drying (MgSO₄) the solution was concentrated and loaded onto a column of silica gel (600 g) set up in diethyl ether:petroleum ether (bp 40-600) (1:2). The column was eluted successively with this mixture, then (1:1) and the diethyl ether to give the title compound (13.88 g). LCMS RT=3.45 min.

### vii) (5R)-3-(6-{[4-(3-Aminophenyl)but-3-ynyl]oxy}hexyl)-5-(2,2-dimethyl-4H-1,3-benzodioxin-6-yl)-1,3-oxazolidin-2-one

To (5R)-3-[6-(but-3-ynyloxy)hexyl]-5-(2,2-dimethyl-4H-1,3-benzodioxin-6-yl)-1,3-oxazolidin-2-one (1.0 g) was added 3-iodoaniline (0.3 ml), acetonitrile (6.0 ml) and triethylamine (3 ml). The resultant mixture was purged with a vigorous stream of nitrogen for 5 min Cuprous iodide (50 mg) and dichlorobis(triphenylphosphine) palladium (50 mg) were added and the reaction mixture was stirred at room temperature under nitrogen for 3 h. The mixture was evaporated to dryness and purified using a 10 g silica Bond Elut cartridge eluting with CH₂Cl₂ and then ether to give the title compound (1.12 g). LCMS RT=3.66 min

### viii) (5R)-3-{6-[4-(3-Aminophenyl)butoxy]hexyl}-5-(2,2-dimethyl-4H-1,3-benzodioxin-6-yl)-1,3-oxazolidin-2-one

(5R)-3-(6-{[4-(3-Aminophenyl)but-3-ynyl]oxy}hexyl)-5-(2,2-dimethyl-4H-1,3-benzodioxin-6-yl)-1,3-oxazolidin-2-one (1.12 g) was stirred with platinum oxide (120 mg) in ethanol (10 ml) and EtOAc (5 ml) under hydrogen for 2 h. The catalyst was removed by filtration through a pad of celite. The filtrate was evaporated to dryness to give the title compound (950 mg). LCMS RT=2.51 min.

### ix) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-(4-fluorophenyl)urea

A solution of (5R)-3-{6-[4-(3-aminophenyl)butoxy]hexyl}-5-(2,2-dimethyl-4H-1,3-benzodioxin-6-yl)-1,3-oxazolidin-2-one (200 mg) in CH₂Cl₂ (4 ml) was reacted with 4-fluorophenylisocyanate (0.046 ml) for 3 h. Methanol (3 ml) was added and the reaction stirred at 20° C. for 60 min. The reaction mixture was concentrated under reduced pressure to give the title compound (202 mg). LCMS RT=4.02 min.

### x) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-(4-fluorophenyl)urea

A solution of N-{3-[4-({6-[(5R)-5-(2,2-dimethyl-4H-1,3-benzodioxin-6-yl)-2-oxo-1,3-oxazolidin-3-yl]hexyl}oxy)butyl]phenyl}-N′-(4-fluorophenyl)urea (202 mg) in TH F (3 ml) was stirred under nitrogen for 5 min. Potassium trimethylsilanolate (204 mg) was added and stirred under nitrogen at 65° C. for 90 min. The reaction mixture was diluted in water (5 ml) and extracted into ethyl acetate (3×20 ml), the resultant organic layers combined, dried (MgSO₄) and the solvent removed under reduced pressure and the residue purified on a Bond Elut Si cartridge (5 g) eluting with 1%, 2%, 3%, 4% MeOH in CH₂Cl₂, followed by 1%, 2%, 3% and 5% ammonia in MeOH in CH₂Cl₂ to give the title compound (138 mg). ES+ve 608 (MH)⁺.

### xi) N-(4-Fluorophenyl)-N′-[3-(4-{[6-({(2R)-2-hydroxy-2-[4-hydroxy-3-(hydroxymethyl)phenyl]ethyl}amino)hexyl]oxy}butyl)phenyl]urea acetate

N-(3-{4-[(6-{[(2R)-2-(2,2-Dimethyl-4H-1,3-benzodioxin-6-yl)-2-hydroxyethyl]amino}hexyl)oxy]butyl}phenyl)-N′-(4-fluorophenyl)urea (138 mg) was stirred with acetic acid (4 ml) and water (2 ml) at 70° C. for 30 min. The resultant mixture was evaporated to dryness and azeotroped with MeOH (2×4 ml) to give the title compound (157 mg). LCMS RT=2.92 min, ES+ve 568 (MH)⁺.

### Example 2

### N-(2,6-Dichlorophenyl)-N′-[3-(4-{[6-({(2R)-2-hydroxy-2-[4-hydroxy-3-(hydroxymethyl)phenyl]ethyl}amino)hexyl]oxy}butyl)phenyl}urea acetate

### i) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-(2,6-dichlorophenyl)urea

was similarly prepared according to Example 1ix. LCMS RT=4.02 min

### ii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-(2,6-dichlorophenyl)urea

was similarly prepared according to Example 1x. LCMS RT=3.05 min

### iii) N-(2,6-Dichlorophenyl)-N′-[3-(4-{[6-({(2R)-2-hydroxy-2-[4-hydroxy-3-(hydroxymethyl)phenyl]ethyl}amino)hexyl]oxy}butyl)phenyl}urea acetate

was similarly prepared according to Example 1xi. LCMS RT=4.02 min

### Example 3

### N-[3-(4-{[6-({(2R)-2-Hydroxy-2-[4-hydroxy-3-(hydroxymethyl)phenyl]ethyl}-amino)hexyl]oxy}butyl)phenyl]-N′-(4-methylphenyl)urea acetate

### i) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-(4-methylphenyl)urea

was similarly prepared according to Example 1ix. LCMS RT=4.09 min

### ii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-(4-methylphenyl)urea

was similarly prepared according to Example 1x. LCMS RT=3.22 min

### iii) N-[3-(4-{[6-({(2R)-2-Hydroxy-2-[4-hydroxy-3-(hydroxymethyl)phenyl]ethyl}amino)hexyl]oxy}butyl)phenyl]-N′-(4-methylphenyl)urea acetate

was similarly prepared according to Example 1ix. LCMS RT=2.82 min. ES+ve 564 (MH)⁺.

### Example 4

### ({[3-(4-{[6-({(2R)-2-Hydroxy-2-[4-hydroxy-3-(hydroxymethyl)phenyl]ethyl}-amino)hexyl]oxy}butyl)anilino]carbonyl}amino)acetic acid acetate

### i) Ethyl[({3-[4-({6-[(5R)-5-(2,2-dimethyl-4H-1,3-benzodioxin-6-yl)-2-oxo-1,3-oxazolidin-3-yl]hexyl}oxy)butyl]anilino}carbonyl)amino]acetate

was similarly prepared according to Example 1ix. LCMS RT=3.72 min

### ii) {[(3-{4-[(6-{[(2R)-2-(2,2-Dimethyl-4H-1,3-benzodioxin-6-yl)-2-hydroxyethyl]amino}hexyl)oxy]butyl}anilino)carbonyl]amino}acetic acid

was similarly prepared according to Example 1x. LCMS RT=2.71 min

### iii) ({[3-(4-{[6-({(2R)-2-Hydroxy-2-[4-hydroxy-3-(hydroxymethyl)phenyl]ethyl}amino)hexyl]oxy}butyl)anilino]carbonyl}amino)acetic acid acetate

was similarly prepared according to Example 1xi. LCMS RT=2.46 min, ES+ve 532 (MH)⁺: ES−ve 530 (M−H)⁻.

### Example 5

### N-[3-(4-{[6-({(2R)-2-Hydroxy-2-[4-hydroxy-3-(hydroxymethyl)phenyl]ethyl}-amino)hexyl]oxy}butyl)phenyl]-N′-[3-(trifluoromethyl)phenyl]urea acetate

### (i) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-[3-(trifluoromethyl)phenyl]urea

was similarly prepared according to Example 1ix. LCMS RT=4.20 min.

### (ii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-[3-(trifluoromethyl)phenyl]urea

was similarly prepared according to Example 1x. LCMS RT=3.31 min. ES+ve 618 (MH)⁺: ES−ve 616 (M−H)⁻.

### (iii) N-[3-(4-{[6-({(2R)-2-Hydroxy-2-[4-hydroxy-3-(hydroxymethyl)phenyl]ethyl}-amino)hexyl]oxy}butyl)phenyl]-N′-[3-(trifluoromethyl)phenyl]urea acetate

was similarly prepared according to Example 1xi. LCMS RT=2.99 min. ES+ve 618 (MH)⁺: ES−ve 616 (M−H)⁻.

### Example 6

### N-(2,6-Dimethylphenyl)-N′-[3-(4-{[6-({(2R)-2-hydroxy-2-[4-hydroxy-3-(hydroxymethyl)phenyl]ethyl}amino)hexyl]oxy}butyl)phenyl]urea acetate

### (i) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-(2,6-dimethylphenyl)urea

was similarly prepared according to Example 1ix. LCMS RT=3.96 min.

### (ii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-(2,6-dimethylphenyl)urea

was similarly prepared according to Example 1x. LCMS RT=3.00 min.

### (iii) N-(2,6-Dimethylphenyl)-N′-[3-(4-{[6-({(2R)-2-hydroxy-2-[4-hydroxy-3-(hydroxymethyl)phenyl]ethyl}amino)hexyl]oxy}butyl)phenyl]urea acetate

was similarly prepared according to Example 1xi. LCMS RT=2.76 min. ES+ve 578 (MH)⁺: ES−ve 576 (M−H)⁻.

### Example 7

### 3-(4-{[6-({(2R)-2-Hydroxy-2-[4-hydroxy-3-(hydroxymethyl)phenyl]ethyl}-amino)hexyl]oxy}butyl)phenyl)-N′-phenylurea acetate

### i) 2-Azido-1-(2,2-dimethyl-4H-1,3-benzodioxin-6-yl)ethanone

2-Bromo-1-(2,2-dimethyl-4H-1,3-benzodioxin-6-yl)ethanone (Glaxo DE 3513885, 1985) (52 g) in DMF (300 ml) was treated with sodium azide (12.24 g) and the mixture was stirred for 2 h at 20° C. The reaction mixture was diluted with EtOAc and washed with water and dried (MgSO₄). The solvent was removed under reduced pressure to give the title compound (39.1 g). TSP+ve 248 (MH)⁺.

### ii) (1R)-2-Azido-1-(2,2-dimethyl-4H-1,3-benzodioxin-6-yl)ethanol

(R)-Tetrahydro-1-methyl-3,3-diphenyl-1H,3H-pyrrolo[1,2-c][1,3,2]oxazaborole solution in toluene (1M, 7.5 ml) was added to THF (75 ml) and the solution was diluted to 0° C. Borane-THF complex (1M solution in THF, 125 ml) was added and the mixture was stirred under nitrogen for 15 min. A solution of 2-azido-1-(2,2-dimethyl-4H-1,3-benzodioxin-6-yl)ethanone (24.7 g) in THF (250 ml) was added dropwise over 1.5 h at 5° C. The mixture was stirred for a further 1 h and then cautiously treated with 2M HCl (100 ml). The reaction mixture was extracted with ether and the organic layer was washed with 2M HCl, NaHCO₃, brine, dried (MgSO₄). The solvent was removed by evaporation and the residue was chromatographed on a Biotage column eluting with ether-petroleum ether (40-60° C.) (1:9; 1:1) to give the title compound (16.99 g). ES+ve 250 (MH)⁺.

### iii) (1R)-2-Amino-1-(2,2-dimethyl-4H-1,3-benzodioxin-6-yl)ethanol

(1R)-2-Azido-1-(2,2-dimethyl-4H-1,3-benzodioxin-6-yl)ethanol (16.99 g) was hydrogenated over 10% Pd—C (1 g) in EtOH (300 ml). The catalyst was collected by filtration, and washed with EtOH. The combined washings were evaporated under reduced pressure and the residue was triturated in ether to give the title compound (5.869). The mother liquors were chromatographed on a Biotage column eluting with toluene:EtOH:aqueous ammonia (85:14:1) to give a further batch of the title compound (5.99 g). LCMS RT=1.68 min, ES+ve 206 (MH-H₂O)⁺.

### iv) 1-{4-[(6-Bromohexyl)oxy]but-1-ynyl}-3-nitrobenzene

A mixture of 1-iodo-3-nitrobenzene (3 g), 1-bromo-6-(3-butynyloxy)hexane (3 g) [Glaxo DE 3513885, 1985], bis(triphenylphosphine)palladium (II) chloride (0.4219), copper (I) iodide (0.114 g) in DMF (10 ml) and diisopropylethylamine (4 ml) was stirred under nitrogen at 20° C. for 5 h. The mixture was concentrated under reduced pressure and the residue was diluted in EtOAc and washed with 2M HCl, NaHCO₃, brine and dried (MgSO₄). The solvent was removed by evaporation and the residue was chromatographed on a Biotage column eluting with ether:petroleum ether (40-60° C.) (1:9) to give the title compound (4.12 g). LCMS RT=4.14 min

### v) 3-{4-[(6-Bromohexyl)oxy]butyl}aniline

1-{4-[(6-Bromohexyl)oxy]but-1-ynyl}-3-nitrobenzene (4.12 g) was hydrogenated over 10% Pd—C (0.3 g) in EtOH (250 ml). The catalyst was collected by filtration and washed with EtOH. The combined filtrate and washings were evaporated under reduced pressure to give the title compound (4.26 g). LCMS RT=3.81 min

### vi) N-(3-{4-[(6-Bromohexyl)oxy]butyl}phenyl)-N′-phenylurea

A solution of 3-{4-[(6-bromohexyl)oxy]butyl}aniline (1 g) in CH₂Cl₂ (10 ml) was reacted with phenylisocyanate (0.4 ml) for 2 h. MeOH (5 ml) was added and the mixture was stirred at 20° C. overnight. The reaction mixture was concentrated under reduced pressure and the residue was purified on a Biotage column eluting with ether:petroleum ether (40-60° C.) (15:85; 3:7; 1:1) to give the title compound (680 mg). ES+ve 447/449 (MH)⁺.

### vii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-phenylurea

A mixture of N-(3-{4-[(6-bromohexyl)oxy]butyl}phenyl)-N′-phenylurea (350 mg) and (1R)-2-amino-1-(2,2-dimethyl-4H-1,3-benzodioxin-6-yl)ethanol (349 mg) in DMF (4 ml) was stirred at 20° C. overnight. The reaction mixture was diluted with CH₂Cl₂ and MeOH and applied to a silica Bond Elut cartridge (10 g). The cartridge was eluted with 3% 2M anhydrous ammonia-MeOH in CH₂Cl₂. The major component was further purified by preparative TLC (4 plates; 20×20 cm) eluting with CH₂Cl₂:MeOH:aqueous ammonia (285:10:5) and extracting the silica with EtOAc:MeOH (2:1) to give the title compound (192 mg). LCMS RT=3.15 min, ES+ve 590 (MH)⁺.

### viii) 3-(4-{[6-({(2R)-2-Hydroxy-2-[4-hydroxy-3-(hydroxymethyl)phenyl]ethyl}amino)hexyl]oxy}butyl)phenyl)-N′-phenylurea acetate

was similarly prepared according to Example 1xi. LCMS RT=2.77 min, ES+ve 550 (MH)⁺; ES−ve 548 (M−H)⁻.

### Example 8

### N-Ethyl-N′-[3-(4-{[6-({(2R)-2-hydroxy-2-[4-hydroxy-3-(hydroxymethyl)phenyl]ethyl}amino)hexyl]oxy}butyl)phenyl)urea acetate

### i) N-(3-{4-[(6-Bromohexyl)oxy]butyl}phenyl)-N′-ethylurea

was similarly prepared according to Example 7vi. ES+ve 399/401 (MH)⁺.

### ii) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-ethylurea

was prepared similarly according to Example 7vii. ES+ve 542 (MH)⁺.

### iii) 3-(4-{[6-({(2R)-2-Hydroxy-2-[4-hydroxy-3-(hydroxymethyl)phenyl]ethyl}amino)hexyl]oxy}butyl)phenyl)-N′-ethylurea acetate

was prepared similarly according to Example 1xi. LCMS RT=2.44 min, ES+ve 502 (MH)⁺

### Example 9

### Ethyl({[3-(4-{[6-({(2R)-2-hydroxy-2-[4-hydroxy-3-(hydroxymethyl)phenyl]ethyl}-amino)hexyl]oxy}butyl)anilino]carbonyl}amino)acetate acetate

### i) Ethyl {[(3-{4-[(6-bromohexyl)oxy]butyl}anilino)carbonyl]amino}acetate

was prepared similarly according to Example 7vi. ES+ve 457/459 (MH)⁺.

### ii) Ethyl {[(3-{4-[(6-{[(2R)-2-(2,2-dimethyl-4H-1,3-benzodioxin-6-yl)-2-hydroxyethyl]amino}hexyl)oxy]butyl}anilino)carbonyl]amino}acetate

was prepared similarly according to Example 7vii. ES+ve 600 (MH)⁺.

### iii) Ethyl({[3-(4-{[6-({(2R)-2-hydroxy-2-[4-hydroxy-3-(hydroxymethyl)phenyl]ethyl}amino)hexyl]oxy}butyl)anilino]carbonyl}amino)acetate acetate

was prepared similarly according to Example 1xi. LCMS RT=2.66 min ES+ve 560 (MH)⁺.

### Example 10

### N-Cyclohexyl-N′-(3-(4-{[6-({(2R)-2-hydroxy-2-[4-hydroxy-3-(hydroxymethyl)phenyl]ethyl}amino)hexyl]oxy}butyl)phenyl)urea acetate

### i) N-(3-{4-[(6-Bromohexyl)oxy]butyl}phenyl)-N′-cyclohexylurea

was prepared similarly according to Example 7vi. ES+ve 453/455 (MH)⁺.

### ii) N-Cyclohexyl-N′-(3-{4-[(6-{[(2R)-2-(2,2-dimethyl-4H-1,3-benzodioxin-6-yl)-2-hydroxyethyl]amino}hexyl)oxy]butyl}phenyl)urea

was prepared similarly according to Example 7vii. ES+ve 596 (MH)⁺.

### iii) N-Cyclohexyl-N′-(3-(4-{[6-({(2R)-2-hydroxy-2-[4-hydroxy-3-(hydroxymethyl)phenyl]ethyl}amino)hexyl]oxy}butyl)phenyl)urea acetate

was prepared similarly according to Example 1xi. LCMS RT 2.62 min ES+ve 556 (MH)⁺.

### Example 11

### N-[4-(4-{[6-({(2R)-2-Hydroxy-2-[4-hydroxy-3-(hydroxymethyl)phenyl]ethyl}amino)hexyl]oxy}butyl)phenyl]-N′-phenylurea acetate

### i) 1-{4-[(6-Bromohexyl)oxy]but-1-ynyl}-4-nitrobenzene

was prepared using methods similar to those described in Example 7iv. tlc (silica) R_{f}=0.42 (10% Et₂O/cyclohexane)

### ii) 1-{4-[(6-Bromohexyl)oxy]butyl}-4-nitrobenzene

was prepared using methods similar to those described in Example 7v. LCMS RT=3.79 min

### iii) N-(4-{4-[(6-Bromohexyl)oxy]butyl}phenyl)-N′-phenylurea

was prepared using methods similar to those described in Example 7vi. ES+ve 447/449 (MH)⁺.

### iv) N-(4-{4-[(6-{[(2R)-2-(2,2-Dimethyl-4H-1,3-benzodioxin-6-yl)-2-hydroxyethyl]amino}hexyl)oxy]butyl}phenyl)-N′-phenylurea

was prepared using methods similar to those described in Example 7vii. LCMS RT=2.96 min, ES+ve 590 (MH)⁺.

### v) N-[4-(4-{[6-({(2R)-2-Hydroxy-2-[4-hydroxy-3-(hydroxymethyl)phenyl]ethyl}amino)hexyl]oxy}butyl)phenyl]-N′-phenylurea acetate

was prepared using methods similar to those described in Example 1xi. LCMS RT=2.71 min, ES+ve 550 (MH)⁺.

### Example 12

### N-Ethyl-N′-[4-(4-{[6-({(2R)-2-hydroxy-2-[4-hydroxy-3-(hydroxymethyl)phenyl]ethyl}amino)hexyl]oxy}butyl)phenyl]urea acetate

### i) N-(4-{4-[(6-Bromohexyl)oxy]butyl}phenyl)-N′-ethylurea

was prepared using methods similar to those described in Example 7vi. ES+ve 399/401 (MH)⁺.

### ii) N-(4-{4-[(6-{[(2R)-2-(2,2-Dimethyl-4H-1,3-benzodioxin-6-yl)-2-hydroxyethyl]amino}hexyl)oxy]butyl}phenyl)-N′-ethylurea

was prepared using methods similar to those described in Example 7vii. ES+ve 542 (MH)⁺.

### iii) N-Ethyl-N′-[4-(4-{[6-({(2R)-2-hydroxy-2-[4-hydroxy-3-(hydroxymethyl)phenyl]ethyl}amino)hexyl]oxy}butyl)phenyl]urea acetate

was prepared using methods similar to those described in Example 1xi. LCMS RT=2.42 min, ES+ve 502 (MH)⁺

### Example 13

### N-[3-(4-{[6-({(2R)-2-Hydroxy-2-[4-hydroxy-3-(hydroxymethyl)phenyl]ethyl}amino)hexyl]oxy}butyl)phenyl]-N′-pyridin-3-ylurea acetate

### i) N-(3-Iodophenyl)-N′-pyridin-3-ylurea hydrochloride

3-Iodophenyl isocyanate (250 mg) and dried 3-aminopyridine (192 mg) were dissolved in CH₂Cl₂ (4 ml) and stirred under nitrogen overnight. MeOH (4 ml) was added and the reaction mixture stirred for 1 h. The solvents were removed in vacuo, the residue was dissolved in EtOAc and 2M HCl and stirred. The solid was removed by filtration, washed with water and air dried to give the title compound (500 mg). LCMS RT=3.05 min.

### ii) N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}-N′-pyridin-3-ylurea

was prepared using methods similar to those described in Example 1vii.

LCMS RT=3.70 min

### iii) N-(3-{4-[(6-{[(2R)-2-(2,2-Dimethyl-4H-1,3-benzodioxin-6-yl)-2-hydroxyethyl]amino}hexyl)oxy]but-1-ynyl}phenyl)-N′-pyridin-3-ylurea

was prepared using methods similar to those described in Example 1x. LCMS RT=2.89 min

### iv) N-(3-{4-[(6-{[(2R)-2-(2,2-Dimethyl-4H-1,3-benzodioxin-6-yl)-2-hydroxyethyl]amino}hexyl)oxy]butyl}phenyl)-N′-pyridin-3-ylurea

N-(3-{4-[(6-{[(2R)-2-(2,2-Dimethyl-4H-1,3-benzodioxin-6-yl)-2-hydroxyethyl]amino}hexyl)oxy]but-1-ynyl}phenyl)-N′-pyridin-3-ylurea (49 mg) was dissolved in EtOH (5 ml) and EtOAc (5 ml) and hydrogenated over 10% Pd/C (5 mg). The catalyst was removed by filtration through celite, and the solvent removed in vacuo. The residue was then dissolved in MeOH and filtered through a cotton wool plug to yield the title compound (36 mg). LCMS RT=2.93 min.

### v) N-[3-(4-{[6-({(2R)-2-Hydroxy-2-[4-hydroxy-3-(hydroxymethyl)phenyl]ethyl}amino)hexyl]oxy}butyl)phenyl]-N′-pyridin-3-ylurea acetate

was prepared using methods similar to those described in Example 1xi. LCMS RT=2.62 min, ES+ve 551 (MH)⁺.

### Example 14

### N-[3-(4-{[6-({(2R)-2-Hydroxy-2-[4-hydroxy-3-(hydroxymethyl)phenyl]ethyl}amino)hexyl]oxy}butyl)phenyl]-N′-pyrimidin-4-ylurea

### i) N-(3-Iodophenyl)-N′-pyrimidin-4-ylurea

A solution of 4-aminopyrimidine (95 mg) in DMF (2 ml) was cooled to 0° C. and treated with a suspension of NaH (60% oil dispersion, 40 mg) in DMF (1 ml). The mixture was stirred under nitrogen for 45 min at 0° C., before 3-iodophenyl isocyanate (245 mg) was added slowly. The reaction mixture was allowed to warm up to room temperature, stirred for 3 h and then water (10 ml) was added. The reaction mixture was then extracted with EtOAc (×3) and the combined organic layers washed with brine (×2), dried (MgSO₄) to yield the title compound (280 mg). LCMS RT=3.40 min.

### ii) N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}-N′-pyrimidin-4-ylurea

was prepared using methods similar to those described in Example 1vii.

LCMS RT=3.79 min.

### iii) N-[3-(4-{[6-({(2R)-2-Hydroxy-2-[4-hydroxy-3-(hydroxymethyl)phenyl]ethyl}amino)hexyl]oxy}but-1-ynyl)phenyl]-N′-pyrimidin-4-ylurea

N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}-N′-pyrimidin-4-ylurea (109 mg, 0.18 mmol) was dissolved in THF (5 ml), treated with potassium trimethylsilanolate (68 mg, 0.53 mmol) and heated to 65° C. under nitrogen. After 5.5 h, the reaction mixture was diluted with MeOH (10 ml) and the solvent removed in vacuo. The residue was dissolved in MeOH (10 ml) and applied to a 10 g SCX cartridge preconditioned with MeOH and eluted with MeOH, 1%, 2% and 2.5% 2M ammonia in MeOH to give an oil. The oil was dissolved in CH₂Cl₂ (2 ml) and MeOH (0.1 ml) and applied to a 1 g silica Bond Elut cartridge preconditioned with and eluted with CH₂Cl₂, 1%, 2%, 3%, 5%, 8% and 10% 2M ammonia in MeOH/CH₂Cl₂ to give the title compound (32 mg). LCMS RT=2.79 min.

### iv) N-[3-(4-{[6-({(2R)-2-Hydroxy-2-[4-hydroxy-3-(hydroxymethyl)phenyl]ethyl}amino)hexyl]oxy}butyl)phenyl]-N′-pyrimidin-4-ylurea

was prepared using methods similar to those described in Example 13iv. LCMS RT=2.85 min, ES+ve 552 (MH)⁺.

### Example 15

### N-[3,5-Bis(trifluoromethyl)phenyl]-N′-[3-(4-{[6-({(2R)-2-hydroxy-2-[4-hydroxy-3-(hydroxymethyl)phenyl]ethyl}amino)hexyl]oxy}butyl)phenyl]urea acetate

### i) N-[3,5-Bis(trifluoromethyl)phenyl]-N′-{3-[4-({6-[(5R)-5-(2,2-dimethyl-4H-1,3-benzodioxin-6-yl)-2-oxo-1,3-oxazolidin-3-yl]hexyl}oxy)butyl]phenyl}urea

was prepared using methods similar to those described in Example 1ix. LCMS RT=4.39 min.

### ii) N-[3,5-Bis(trifluoromethyl)phenyl]-N′-(3-{4-[(6-{[(2R)-2-(2,2-dimethyl-4H-1,3-benzodioxin-6-yl)-2-hydroxyethyl]amino}hexyl)oxy]butyl}phenyl)urea

was prepared using methods similar to those described in Example 1x. LCMS RT=3.40 min.

### iii) N-[3,5-Bis(trifluoromethyl)phenyl]-N′-[3-(4-{[6-({(2R)-2-hydroxy-2-[4-hydroxy-3-(hydroxymethyl)phenyl]ethyl}amino)hexyl]oxy}butyl)phenyl]urea acetate

was prepared using methods similar to those described in Example 1xi. LCMS RT=3.36 min, ES−ve 684 (M−H)⁻.

### Example 16

### N-Cyclohexyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

### i) (5R)-3-[6-({4-[3-(Aminomethyl)phenyl]but-3-ynyl}oxy)hexyl]-5-(2,2-dimethyl-4H-1,3-benzodioxin-6-yl)-1,3-oxazolidin-2-one

was prepared using methods similar to those described in Example 1vii. LCMS RT=2.77 min.

### ii) (5R)-3-(6-{4-[3-(Aminomethyl)phenyl]butoxy}hexyl)-5-(2,2-dimethyl-4H-1,3-benzodioxin-6-yl)-1,3-oxazolidin-2-one

was prepared using methods similar to those described in Example 1viii. LCMS RT=2.98 min.

### iii) N-Cyclohexyl-N′-{3-[4-({6-[(5R)-5-(2,2-dimethyl-4H-1,3-benzodioxin-6-yl)-2-oxo-1,3-oxazolidin-3-yl]hexyl}oxy)butyl]benzyl}urea

was prepared using methods similar to those described in Example 1ix. LCMS RT=3.93 min.

### iv) N-Cyclohexyl-N′-(3-{4-[(6-{[(2R)-2-(2,2-dimethyl-4H-1,3-benzodioxin-6-yl)-2-hydroxyethyl]amino}hexyl)oxy]butyl}benzyl)urea

was prepared using methods similar to those described in Example 1x. LCMS RT=2.92 min.

### v) N-Cyclohexyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

was prepared using methods similar to those described in Example 1xi. LCMS RT=2.69 min, ES+ve 570 (MH)⁺.

### Example 17

### N-Ethyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

### i) N-{3-[4-({6-[(5R)-5-(2,2-Dimethyl-4H-1,3-benzodioxin-6-yl)-2-oxo-1,3-oxazolidin-3-yl]hexyl}oxy)butyl]benzyl}-N′-ethylurea

was prepared using methods similar to those described in Example 1ix.

LCMS RT=3.62 min.

### ii) N-(3-{4-[(6-{[(2R)-2-(2,2-Dimethyl-4H-1,3-benzodioxin-6-yl)-2-hydroxyethyl]amino}hexyl)oxy]butyl}benzyl)-N′-ethylurea

was prepared using methods similar to those described in Example 1x. LCMS RT=2.68 min.

### iii) N-Ethyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

was prepared using methods similar to those described in Example 1xi. LCMS RT=2.55 min, ES+ve 516 (MH)⁺.

### Example 18

### N-[3-(4-{[6-({(2R)-2-Hydroxy-2-[4-hydroxy-3-(hydroxymethyl)phenyl]ethyl}amino)hexyl]oxy}butyl)benzyl]urea acetate

### i) Ethyl N-[({3-[4-({6-[(5R)-5-(2,2-dimethyl-4H-1,3-benzodioxin-6-yl)-2-oxo-1,3-oxazolidin-3-yl]hexyl}oxy)butyl]benzyl}amino)carbonyl]glycinate

was prepared using methods similar to those described in Example 1ix. LCMS RT=3.55 min.

### ii) N-(3-{4-[(6-[{(2R)-2-(2,2-Dimethyl-4H-1,3-benzodioxin-6-yl)-2-hydroxyethyl]amino}hexyl)oxy]butyl}benzyl)urea

was prepared using methods similar to those described in Example 1x. LCMS RT=2.59 min.

### iii) N-[3-(4-{[6-({(2R)-2-Hydroxy-2-[4-hydroxy-3-(hydroxymethyl)phenyl]ethyl}amino)hexyl]oxy}butyl)benzyl]urea acetate

was prepared using methods similar to those described in Example 1xi. LCMS RT=2.66 min, ES+ve 488 (MH)⁺.

### Example 19

### N-(4-Fluorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea acetate

### i) N-{3-[4-({6-[(5R)-5-(2,2-Dimethyl-4H-1,3-benzodioxin-6-yl)-2-oxo-1,3-oxazolidin-3-yl]hexyl}oxy)butyl]benzyl}-N′-(4-fluorophenyl)urea

was prepared using methods similar to those described in Example 1ix. LCMS RT=3.84 min.

### ii) N-(3-{4-[(6-{[(2R)-2-(2,2-Dimethyl-4H-1,3-benzodioxin-6-yl)-2-hydroxyethyl]amino}hexyl)oxy]butyl}benzyl)-N′-(4-fluorophenyl)urea

was prepared using methods similar to those described in Example 1x. LCMS RT=3.09 min.

### iii) N-(4-Fluorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea acetate

was prepared using methods similar to those described in Example 1xi. LCMS RT=2.72 min, ES+ve 582 (MH)⁺.

### Example 20

### N-(3-Chlorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea acetate

### i) N-(3-Chlorophenyl)-N′-{3-[4-({6-[(5R)-5-(2,2-dimethyl-4H-1,3-benzodioxin-6-yl)-2-oxo-1,3-oxazolidin-3-yl]hexyl}oxy)butyl]benzyl}urea

was prepared using methods similar to those described in Example 1ix. LCMS RT=4.00 min.

### ii) N-(3-Chlorophenyl)-N′-(3-{4-[(6-{[(2R)-2-(2,2-dimethyl-4H-1,3-benzodioxin-6-yl)-2-hydroxyethyl]amino}hexyl)oxy]butyl}benzyl)urea

was prepared using methods similar to those described in Example 1x. LCMS RT=3.05 min.

### iii) N-(3-Chlorophenyl)-N′-[3-(4-{[6-({(2R)-2-hydroxy-2-[4-hydroxy-3-(hydroxymethyl)phenyl]ethyl}amino)hexyl]oxy}butyl)benzyl]urea acetate

was prepared using methods similar to those described in Example 1xi. LCMS RT=2.96 min, ES+ve 598, 600 (MH)⁺.

### Example 21

### N-Benzyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

### i) N-Benzyl-N′-{3-[4-({6-[(5R)-5-(2,2-dimethyl-4H-1,3-benzodioxin-6-yl)-2-oxo-1,3-oxazolidin-3-yl]hexyl}oxy)butyl]benzyl}urea

was prepared using methods similar to those described in Example 1ix. LCMS RT=3.75 min.

### ii) N-Benzyl-N′-(3-{4-[(6-{[(2R)-2-(2,2-dimethyl-4H-1,3-benzodioxin-6-yl)-2-hydroxyethyl]amino}hexyl)oxy]butyl}benzyl)urea

was prepared using methods similar to those described in Example 1x. LCMS RT=3.04 min

### iii) N-Benzyl-N′-[3-(4-{[6-({(2R)-2-hydroxy-2-[4-hydroxy-3-(hydroxymethyl)phenyl]ethyl}amino)hexyl]oxy}butyl)benzyl]urea acetate

as prepared using methods similar to those described in Example 1xi. LCMS RT=2.65 min, ES+ve 578 (MH)⁺.

### Example 22

### N-({[2-(4-{[6-({(2R)-2-Hydroxy-2-[4-hydroxy-3-(hydroxymethyl)phenyl]ethyl}amino)hexyl]oxy}butyl)benzyl]amino}carbonyl)glycine acetate

### i) Ethyl N-{[(2-bromobenzyl)amino]carbonyl}glycinate

was prepared using methods similar to those described in Example 13i. LCMS RT=2.84 min.

### ii) N-{[(2-Bromobenzyl)amino]carbonyl}glycine

To a stirred solution of ethyl N-{[(2-bromobenzyl)amino]carbonyl}glycinate (200 mg) in THF (3 ml) and MeOH (0.5 ml) was added potassium-trimethylsilanolate (81 mg) and the reaction mixture stirred at room temperature for 3 h. After this time, the solvent was removed in vacuo and the residue was dissolved in water (10 ml) and extracted with EtOAc (3×25 ml). The combined organic layers were dried (MgSO₄) and the solvent removed in vacuo to give the title compound (115 mg). LCMS RT=2.64 min.

### iii) 1-[2-(4-{6-[5-(2,2-Dimethyl-4H-benzo[1,3]dioxin-6-yl)-2-oxo-oxazolidin-3-yl]-hexyloxy}-butyl)-benzyl]-3-(2-oxo-2-pyrrolidin-1-yl-ethyl)-urea

To a stirred solution of N-{[(2-bromobenzyl)amino]carbonyl}glycine (175 mg) and tetrakis(triphenylphosphine)palladium (0) (20 mg) in pyrrolidine (2 ml) under nitrogen, was added a solution of (5R)-3-[6-(but-3-ynyloxy)hexyl]-5-(2,2-dimethyl-4H-1,3-benzodioxin-6-yl)-1,3-oxazolidin-2-one (example 1vi) (222 mg) in pyrrolidine (4 ml) and the reaction mixture was heated to 80° C. After 5 h, water (10 ml) was added and extracted with EtOAc (3×25 ml), the combined organic layers dried (MgSO₄) and the solvent removed in vacuo. The residue was dissolved in CH₂Cl₂ (8 ml) and applied to a 10 g silica Bond Elut cartridge preconditioned with CH₂Cl₂ and eluted with CH₂Cl₂, EtOAc and 10% MeOH in CH₂Cl₂ to give the title compound (370 mg). LCMS RT=3.46 min.

### iv) 1-[2-(4-{6-[5-(2,2-Dimethyl-4H-benzo[1,3]dioxin-6-yl)-2-oxo-oxazolidin-3-yl]-hexyloxy}-but-1-ynyl)-benzyl]-3-(2-oxo-2-pyrrolidin-1-yl-ethyl)-urea

was prepared using methods similar to those described in Example 1viii. LCMS RT=3.50 min.

### v) N-{[(2-{4-[(6-{[(2R)-2-(2,2-Dimethyl-4H-1,3-benzodioxin-6-yl)-2-hydroxyethyl]amino}hexyl)oxy]butyl}benzyl)amino]carbonyl}glycine

was prepared using methods similar to those described in Example 1x. LCMS RT=2.77 min.

### vi) N-({[2-(4-{[6-({(2R)-2-Hydroxy-2-[4-hydroxy-3-(hydroxymethyl)phenyl]ethyl}amino)hexyl]oxy}butyl)benzyl]amino}carbonyl)glycine acetate

was prepared using methods similar to those described in Example 1xi. LCMS RT=2.57 min, ES+ve 546 (MH)⁺.

### Example 23

### N-{2-[3-(4-{[6-({(2R)-2-Hydroxy-2-[4-hydroxy-3-(hydroxymethyl)phenyl]ethyl}amino)hexyl]oxy}butyl)phenyl]ethyl}-N′-phenylurea acetate

### i) (5R)-3-[6-({4-[3-(2-Aminoethyl)phenyl]but-3-ynyl}oxy)hexyl]-5-(2,2-dimethyl-4H-1,3-benzodioxin-6-yl)-1,3-oxazolidin-2-one

To a stirred solution of 2-(3-bromophenyl)ethanamine (500 mg) and tetrakis (triphenylphosphine)palladium (0) (60 mg) in pyrrolidine (4 ml) under nitrogen, was added a solution of (5R)-3-[6-(but-3-ynyloxy)hexyl]-5-(2,2-dimethyl-4H-1,3-benzodioxin-6-yl)-1,3-oxazolidin-2-one (example 1vi) (912 mg) in pyrrolidine (4 ml) and the reaction mixture was heated to 80° C. After 18 h, water (10 ml) was added and extracted with EtOAc (3×25 ml), the combined organic layers dried (MgSO₄) and the solvent removed in vacuo. The residue was dissolved in CH₂Cl₂ (25 ml) and applied to a Biotage cartridge (40 g) and eluted with CH₂Cl₂, EtOAc and CH₂Cl₂:EtOH:aq NH₃ (100:8:1) to give the title compound (668 mg). LCMS RT=3.09 min.

### ii) (5R)-3-(6-{4-[3-(2-Aminoethyl)phenyl]butoxy}hexyl)-5-(2,2-dimethyl-4H-1,3-benzodioxin-6-yl)-1,3-oxazolidin-2-one

was prepared using methods similar to those described in Example 1 viii. LCMS RT=3.14 min.

### iii) N-(2-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}ethyl)-N′-phenylurea

was prepared using methods similar to those described in Example 1ix. LCMS RT=3.98 min.

### iv) N-[2-(3-{4-[(6-{[(2R)-2-(2,2-Dimethyl-4H-1,3-benzodioxin-6-yl)-2-hydroxyethyl]amino}hexyl)oxy]butyl}phenyl)ethyl]-N′-phenylurea

was prepared using methods similar to those described in Example 1x. LCMS RT=3.27 min.

### v) N-{2-[3-(4-{[6-({(2R)-2-Hydroxy-2-[4-hydroxy-3-(hydroxymethyl)phenyl]ethyl}amino)hexyl]oxy}butyl)phenyl]ethyl}-N′-phenylurea acetate

was prepared using methods similar to those described in Example 1xi. LCMS RT=2.98 min, ES+ve 578 (MH)⁺.

### Example 24

### N-[3-(4-{[6-({(2R)-2-Hydroxy-2-[4-hydroxy-3-(hydroxymethyl)phenyl]ethyl}amino)hexyl]oxy}butyl)phenyl]urea acetate

### i) N-(3-Iodophenyl)urea

A suspension of sodium cyanate (6.5 g) in water (50 ml) was slowly added to a solution of 3-iodoaniline (6 ml) in 50% aqueous acetic acid (40 ml) and the mixture was stirred for 3 h at 20° C. Water (300 ml) was added and the solid was collected by filtration. The solid washed with water, air-dried and triturated in ether to give the title compound (11.93 g). ES+ve 263 (MH)⁺.

### ii) N-(3-{4-[(6-Bromohexyl)oxy]but-1-ynyl}phenyl)urea

A mixture of N-(3-iodophenyl)urea (1.05 g), 6-bromohexyl but-3-ynyl ether (1 g) [Glaxo DE3513885], bis(triphenylphosphine)palladium (II) chloride (140 mg), copper (I) iodide (38 mg) in DMF (5 ml) and diisopropylethylamine (2 ml) was stirred under nitrogen at 20° C. for 15 h. The mixture was diluted with EtOAc and washed with 2M HCl, NaHCO₃, brine and dried (MgSO₄). The solvent was removed by evaporation and the residue was chromatographed on a Biotage column eluting with CH₂Cl₂ and MeOH:CH₂Cl₂ (1:49) to give the title compound (656 mg). ES+ve 367/369 (MH)⁺.

### iii) N-(3-{4-[(6-Bromohexyl)oxy]butyl}phenyl)urea

N-(3-{4-[(6-bromohexyl)oxy]but-1-ynyl}phenyl)urea (650 mg) was hydrogenated over platinum oxide (70 mg) in EtOAc (75 ml) for 16 h. The catalyst was collected by filtration, washed with EtOAc and the combined filtrate and washings were evaporated under reduced pressure to give mainly the title compound but contaminated with some partially hydrogenated product. ES+ve 369/371/373 (MH)⁺.

### iv) N-(3-{4-[(6-{[(2R)-2-(2,2-Dimethyl-4H-1,3-benzodioxin-6-yl)-2-hydroxyethyl]amino}hexyl)oxy]butyl}phenyl)urea

The above product (680 mg) was reacted with (1R)-2-amino-1-(2,2-dimethyl-4H-1,3-benzodioxin-6-yl)ethanol (470 mg) in DMF (4 ml) overnight. The mixture was diluted with EtOAc and washed with water, brine, dried (MgSO₄). The solution was evaporated and the residue was purified on a Biotage column eluting with 2M anhydrous ammonia in MeOH:CH₂Cl₂ (1:24) to give mainly the title compound contaminated with some unsaturated material (400 mg). ES+ve 512/514 (MH)⁺.

### iv) N-[3-(4-{[6-({(2R)-2-Hydroxy-2-[4-hydroxy-3-(hydroxymethyl)phenyl]ethyl}amino)hexyl]oxy}butyl)phenyl]urea acetate

The above mixture was hydrogenated over platinum oxide (85 mg) in EtOH (75 ml) for 3 h. The catalyst was collected by filtration and washed with EtOH. The combined filtrate and washings were evaporated under reduced pressure to give the title compound (350 mg). ES+ve 514 (MH)⁺.

### Example 25

### N-[3-(3-{[7-({(2R)-2-Hydroxy-2-[4-hydroxy-3-(hydroxymethyl)phenyl]ethyl}amino)heptyl]oxy}propyl)phenyl]urea acetate

### i) 7-bromoheptyl prop-2-ynyl ether

was prepared using methods similar to those described in Example 1v.

LCMS RT=3.63 min.

### ii) N-(3-{3-[(7-Bromoheptyl)oxy]prop-1-ynyl}phenyl)urea and N-(3-{3-[(7-Iodoheptyl)oxy]prop-1-ynyl}phenyl)urea

A mixture of N-(3-iodophenyl)urea (524 mg), 7-bromoheptyl prop-2-ynyl ether (490 mg), bis(triphenylphosphine)palladium (II) chloride (70 mg), copper (I) iodide (19 mg) and N,N-diisopropylethylamine (1.05 ml) in DMF (5 ml) was stirred under nitrogen at 20° C. for 18 h. The mixture was then diluted in EtOAc and washed with 2M HCl, NaHCO₃, brine and dried (MgSO₄). The solution was concentrated in vacuo and the residue was purified by chromatography (Biotage, 40 g) eluting with CH₂Cl₂-MeOH (99:1) to give the title compounds (421 mg) as a 55:45 ratio respectively. LCMS RT=3.42 and 3.55 min.

### iii) N-(3-{3-[(7-{[(2R)-2-(2,2-Dimethyl-4H-1,3-benzodioxin-6-yl)-2-hydroxyethyl]amino}heptyl)oxy]prop-1-ynyl}phenyl)urea

The above mixture (421 mg) was reacted with (1R)-2-amino-1-(2,2-dimethyl-4H-1,3-benzodioxin-6-yl)ethanol (514 mg) in DMF (5 ml) for 18 h. The mixture was diluted with EtOAc and washed with water, brine and dried (MgSO₄). The solution was concentrated in vacuo and the residue was purified by chromatography (Biotage, 40 g) eluting with CH₂Cl₂ and then CH₂Cl₂-MeOH: 2M NH₃/MeOH (97:2:1), (95:3:2), (95:4:1) and (90:6:4) to give the title compound (355 mg). LCMS RT=2.62 min

### iv) N-(3-{3-[(7-{[(2R)-2-(2,2-Dimethyl-4H-1,3-benzodioxin-6-yl)-2-hydroxyethyl]amino}heptyl)oxy]propyl}phenyl)urea

Prepared using methods similar to those described in Example 1 viii)

LCMS RT=2.60 min

### v) N-[3-(3-{[7-({(2R)-2-Hydroxy-2-[4-hydroxy-3-(hydroxymethyl)phenyl]ethyl}amino)heptyl]oxy}propyl)phenyl]urea acetate

Prepared using methods similar to those described in Example 1 xi)

LCMS RT=2.37 min, ES+ve 474 (MH)⁺

### Example 26

### N-[3-(5-{[5-({(2R)-2-Hydroxy-2-[4-hydroxy-3-(hydroxymethyl)phenyl]ethyl}amino)pentyl]oxy}pentyl)phenyl]urea acetate

### i) N-(3-{5-[(5-Bromopentyl)oxy]pent-1-ynyl}phenyl)urea and N-(3-{5-[(5-iodopentyl)oxy]pent-1-ynyl}phenyl)urea

Prepared using methods similar to those described in Example 25i)

Product ratio=66:34. LCMS RT=3.38 and 3.50 min.

### ii) N-(3-{5-[(5-{[(2R)-2-(2,2-Dimethyl-4H-1,3-benzodioxin-6-yl)-2-hydroxyethyl]amino}pentyl)oxy]pent-1-ynyl}phenyl)urea

Prepared using methods similar to those described in Example 25 ii)

LCMS RT=2.52 min.

### iii) N-(3-{5-[(5-{[(2R)-2-(2,2-Dimethyl-4H-1,3-benzodioxin-6-yl)-2-hydroxyethyl]amino}pentyl)oxy]pentyl}phenyl)urea

Prepared using methods similar to those described in Example 1 viii)

LCMS RT=2.56 min

### iv) N-[3-(5-{[5-({(2R)-2-Hydroxy-2-[4-hydroxy-3-(hydroxymethyl)phenyl]ethyl}amino)pentyl]oxy}pentyl)phenyl]urea acetate

Prepared using methods similar to those described in Example 1 xi)

LCMS RT=2.39 min, ES+ve 474 (MH)⁺

### Example 27

### N-[3-(5-{[6-({(2R)-2-Hydroxy-2-[4-hydroxy-3-(hydroxymethyl)phenyl]ethyl}amino)hexyl]oxy}pentyl)phenyl]urea acetate

### i) N-(3-{5-[(6-Bromohexyl)oxy]pent-1-ynyl}phenyl)urea and N-(3-{5-[(6-iodohexyl)oxy]pent-1-ynyl}phenyl)urea

Prepared using methods similar to those described in Example 25 i)

Product ratio=66:34. LCMS RT=3.64 and 3.76 min.

### ii) N-(3-{5-[(6-{[(2R)-2-(2,2-Dimethyl-4H-1,3-benzodioxin-6-yl)-2-hydroxyethyl]amino}hexyl)oxy]pent-1-ynyl}phenyl)urea

Prepared using methods similar to those described in Example 25 ii)

### iii) N-(3-{5-[(6-{[(2R)-2-(2,2-Dimethyl-4H-1,3-benzodioxin-6-yl)-2-hydroxyethyl]amino}hexyl)oxy]pentyl}phenyl)urea

Prepared using methods similar to those described in Example 1 viii)

LCMS RT=2.71 min

### iv) N-[3-(5-{[6-({(2R)-2-Hydroxy-2-[4-hydroxy-3-(hydroxymethyl)phenyl]ethyl}amino)hexyl]oxy}pentyl)phenyl]urea acetate

Prepared using methods similar to those described in Example 1 xi)

LCMS RT=2.53 min, ES+ve 488 (MH)⁺

### Example 28

### N-[3-(4-{[6-({(2R)-2-Hydroxy-2-[4-hydroxy-3-(hydroxymethyl)phenyl]ethyl}amino)hexyl]oxy}butyl)-5-(trifluoromethyl)phenyl]urea acetate

### i) N-[3-Bromo-5-(trifluoromethyl)phenyl]urea

was prepared using methods similar to those described in Example 24i)

LCMS RT=3.20 min

### ii) N-[3-{4-[(6-Bromohexyl)oxy]but-1-ynyl}-5-(trifluoromethyl)phenyl]urea

was prepared using methods similar to those described in Example 25i)

LCMS RT=3.84 min

### iii) N-[3-{4-[(6-{[(2R)-2-(2,2-Dimethyl-4H-1,3-benzodioxin-6-yl)-2-hydroxyethyl]amino}hexyl)oxy]but-1-ynyl}-5-(trifluoromethyl)phenyl]urea

was prepared using methods similar to those described in Example 25ii)

LCMS RT=2.86 min

### iv) N-[3-{4-[(6-{[(2R)-2-(2,2-Dimethyl-4H-1,3-benzodioxin-6-yl)-2-hydroxyethyl]amino}hexyl)oxy]butyl}-5-(trifluoromethyl)phenyl]urea

was prepared using methods similar to those described in Example 1viii)

LCMS RT=2.75 min

### v) N-[3-(4-{[6-({(2R)-2-Hydroxy-2-[4-hydroxy-3-(hydroxymethyl)phenyl]ethyl}amino)hexyl]oxy}butyl)-5-(trifluoromethyl)phenyl]urea acetate

was prepared using methods similar to those described in Example 1 xi)

LCMS RT=2.62 min, ES+ve 542 (MH)⁺.

### Example 29

### N-[3-(4-{[6-({(2R)-2-Hydroxy-2-[4-hydroxy-3-(hydroxymethyl)phenyl]ethyl}amino)hexyl]oxy}butyl)-5-methylphenyl]urea acetate

### i) (5R)-5-(2,2-Dimethyl-4H-1,3-benzodioxin-6-yl)-3-(6-{[4-(3-methyl-5-nitrophenyl)but-3-ynyl]oxy}hexyl)-1,3-oxazolidin-2-one

To a degassed solution of anhydrous tetrahydrofuran (4 ml) and triethylamine (0.5 ml) was added 1-bromo-3-methyl-5-nitrobenzene (135 mg), dichloro bis(triphenylphosphine) palladium (II) (31 mg) and cuprous iodide (15 mg). The resultant mixture was then purged with nitrogen and heated to 70°. After 10 min, a solution of (5R)-3-[6-(but-3-ynyloxy)hexyl]-5-(2,2-dimethyl-4H-1,3-benzodioxin-6-yl)-1,3-oxazolidin-2-one (250 mg) in anhydrous degassed THF (1 ml) was added and the reaction mixture stirred at 700 for 6 h. The cooled reaction mixture was evaporated to dryness and the residue purified using a 10 g silica Bond Elut cartridge, eluting with CH₂Cl₂ and then 0-50% ethyl acetate in cyclohexane gradient to give the title compound (92 mg). LCMS RT=3.94 min

### ii) (5R)-3-{6-[4-(3-Amino-5-methylphenyl)butoxy]hexyl}-5-(2,2-dimethyl-4H-1,3-benzodioxin-6-yl)-1,3-oxazolidin-2-one

(5R)-5-(2,2-Dimethyl-4H-1,3-benzodioxin-6-yl)-3-(6-{[4-(3-methyl-5-nitrophenyl)but-3-ynyl]oxy}hexyl)-1,3-oxazolidin-2-one (92 mg) was stirred with platinum oxide (15 mg) in ethanol (4 ml) and EtOAc (few drops) under hydrogen for 3 h. The catalyst was removed by filtration through a pad of celite. The filtrate was evaporated to dryness and the residue purified using a 1 g silica Bond Elut cartridge, eluting with CH₂Cl₂ and then 0-60% ethyl acetate in cyclohexane gradient to give the title compound (64 mg). LCMS RT=3.58 min.

### iii) N-{3-[4-({6-[(5R)-5-(2,2-Dimethyl-4H-1,3-benzodioxin-6-yl)-2-oxo-1,3-oxazolidin-3-yl]hexyl}oxy)butyl]-5-methylphenyl}urea

A suspension of potassium cyanate (127 mg) in water (3 ml) was slowly added to a solution of (5R)-3-{6-[4-(3-amino-5-methylphenyl)butoxy]hexyl}-5-(2,2-dimethyl-4H-1,3-benzodioxin-6-yl)-1,3-oxazolidin-2-one (400 mg) in glacial acetic acid (3 ml) containing water (1.5 ml) at −0° C. under nitrogen. The mixture was warmed to room temperature over ˜2 h and then stirred at room temperature for 20 min. The reaction mixture was evaporated to dryness and the residue purified using a 10 g silica Bond Elut cartridge, eluting with CH₂Cl₂ and then 0-100% ethyl acetate-cyclohexane gradient to give the title compound (299 mg). LCMS RT=3.57 min

### iv) N-(3-{4-[(6-{[(2R)-2-(2,2-Dimethyl-4H-1,3-benzodioxin-6-yl)-2-hydroxyethyl]amino}hexyl)oxy]butyl}-5-methylphenyl)urea

To a solution of N-{3-[4-({6-[(5R)-5-(2,2-dimethyl-4H-1,3-benzodioxin-6-yl)-2-oxo-1,3-oxazolidin-3-yl]hexyl}oxy)butyl]-5-methylphenyl}urea (66 mg) in anhydrous THF (2.5 ml) was added potassium trimethylsilanolate (61 mg). The reaction was stirred under nitrogen at 65° C. for 105 min. The cooled reaction mixture was then diluted with water and extracted into ethyl acetate (×4), the resultant organic layers combined, dried (MgSO₄) and filtered. The filtrate was evaporated to dryness and the residue purified using a 1 g silica Bond Elut cartridge, eluting with CH₂Cl₂, 0-100% ethyl acetate in cyclohexane gradient followed by 0-8% methanol in dichloromethane (and trace of ammonia solution) gradient to give the title compound (26 mg). LCMS RT=2.79 min

### v) N-[3-(4-{[6-({(2R)-2-Hydroxy-2-[4-hydroxy-3-(hydroxymethyl)phenyl]ethyl}amino)hexyl]oxy}butyl)-5-methylphenyl]urea acetate

N-(3-{4-[(6-{[(2R)-2-(2,2-Dimethyl-4H-1,3-benzodioxin-6-yl)-2-hydroxyethyl]amino}hexyl)oxy]butyl}-5-methylphenyl)urea (26 mg) was stirred with glacial acetic acid (1 ml) and water (0.5 ml) at 80° C. for 50 min. The resultant reaction mixture was cooled and evaporated to dryness and the residue azeotroped with MeOH to give the title compound (29 mg). LCMS RT=2.55 min, ES+ve 488 (MH)⁺.

### Example 30

### 5-(4-{[6-({(2R)-2-Hydroxy-2-[4-hydroxy-3-(hydroxymethyl)phenyl]ethyl}amino)hexyl]oxy}butyl)-1,3-dihydro-2H-benzimidazol-2-one acetate

### i) N-Benzyl-4-iodo-2-nitroaniline

A mixture of benzylamine (0.84 ml), diisopropylethylamine (1.33 ml) and 1-fluoro-4-iodo-2-nitrobenzene (1.02 g) in dichloromethane (10 ml) was stirred for 15 h at 20° C. The mixture was diluted with dichloromethane and washed with aqueous 2M HCl, NaHCO₃ solution, dried (MgSO₄) and filtered. The filtrate was evaporated to give the title compound (1.25 g) LCMS RT=4.01 min

### ii) (5R)-3-[6-({4-[4-(Benzylamino)-3-nitrophenyl]but-3-ynyl}oxy)hexyl]-5-(2,2-dimethyl-4H-1,3-benzodioxin-6-yl)-1,3-oxazolidin-2-one was prepared using methods similar to those described in Example 1vii)

LCMS RT=3.62 min

### iii) (5R)-3-{6-[4-(3,4-Diaminophenyl)butoxy]hexyl}-5-(2,2-dimethyl-4H-1,3-benzodioxin-6-yl)-1,3-oxazolidin-2-one

was prepared using methods similar to those described in Example 1viii)

LCMS RT=3.21 min

### iv) 5-[4-({6-[(5R)-5-(2,2-Dimethyl-4H-1,3-benzodioxin-6-yl)-2-oxo-1,3-oxazolidin-3-yl]hexyl}oxy)butyl]-1,3-dihydro-2H-benzimidazol-2-one

A solution of (5R)-3-{6-[4-(3,4-diaminophenyl)butoxy]hexyl}-5-(2,2-dimethyl-4H-1,3-benzodioxin-6-yl)-1,3-oxazolidin-2-one (176 mg) and carbonyldiimidazole (206 mg) in THF (5 ml) was stirred at 20° C. for 16 h. The mixture was purified on a 10 g Bond Elut cartridge eluting with dichloromethane-MeOH (1:0 to 19:1) to give the title compound (71 mg)

LCMS RT=3.62 min.

### v) 5-(4-{[6-({(2R)-2-Hydroxy-2-[4-hydroxy-3-(hydroxymethyl)phenyl]ethyl}amino)hexyl]oxy}butyl)-1,3-dihydro-2H-benzimidazol-2-one was prepared using methods similar to those described in Example 14iii) LCMS RT=2.44 min.

### vi) 5-(4-{[6-({(2R)-2-Hydroxy-2-[4-hydroxy-3-(hydroxymethyl)phenyl]ethyl}amino)hexyl]oxy}butyl)-1,3-dihydro-2H-benzimidazol-2-one acetate

was prepared using methods similar to those described in Example 1 xi)

LCMS RT=2.44 min, ES+ve 472 (MH)⁺.

### Example 31

### N-Benzoyl-N′-[3-(4-{[6-({(2R)-2-hydroxy-2-[4-hydroxy-3-(hydroxymethyl)phenyl]ethyl}amino)hexyl]oxy}butyl)phenyl]urea

### i) N-Benzoyl-N′-(3-iodophenyl)urea

3-Iodoaniline (0.5 g) in dichloromethane (5 ml) was treated with benzoyl isocyanate (0.34 g) in dichloromethane (7 ml) and the mixture was stirred at 20° C. for 15 h. MeOH (10 ml) was added and after 4 h the solid was collected by filtration and dried to give the title compound (0.59 g) LCMS RT=3.76 min

### ii) N-Benzoyl-N′-(3-{4-[(6-bromohexyl)oxy]but-1-ynyl}phenyl)urea

was prepared using methods similar to those described in Example 1vii)

LCMS RT=4.11 min

### iii) N-Benzoyl-N′-(3-{4-[(6-{[(2R)-2-(2,2-dimethyl-4H-1,3-benzodioxin-6-yl)-2-hydroxyethyl]amino}hexyl)oxy]but-1-ynyl}phenyl)urea

was prepared using methods similar to those described in Example 7vii)

LCMS RT=3.17 min

### iv) N-Benzoyl-N′-[3-(4-{[6-({(2R)-2-hydroxy-2-[4-hydroxy-3-(hydroxymethyl)phenyl]ethyl}amino)hexyl]oxy}butyl)phenyl]urea

was prepared using methods similar to those described in Example 14iii)

LCMS RT=2.93 min, ES+ve 578 (MH)⁺.

### Example 32

### N-[2-(4-{[6-({(2R)-2-Hydroxy-2-[4-hydroxy-3-(hydroxymethyl)phenyl]ethyl}amino)hexyl]oxy}butyl)phenyl]-N′-phenylurea acetate

### i) N-(2-Iodophenyl)-N′-phenylurea

was prepared using methods similar to those described in Example 31i)

LCMS RT=3.61 min

### ii) N-(2-{4-[(6-Bromohexyl)oxy]but-1-ynyl}phenyl)-N′-phenylurea

was prepared using methods similar to those described in Example 1vii)

LCMS RT=3.61 min

### iii) N-(2-{4-[(6-{[(2R)-2-(2,2-Dimethyl-4H-1,3-benzodioxin-6-yl)-2-hydroxyethyl]amino}hexyl)oxy]but-1-ynyl}phenyl)-N′-phenylurea

was prepared using methods similar to those described in Example 7vii)

LCMS RT=2.83 min

### iv) N-(2-{4-[(6-{[(2R)-2-(2,2-Dimethyl-4H-1,3-benzodioxin-6-yl)-2-hydroxyethyl]amino}hexyl)oxy]butyl}phenyl)-N′-phenylurea

was prepared using methods similar to those described in Example 1viii)

LCMS RT=2.79 min

### v) N-[2-(4-{[6-({(2R)-2-hydroxy-2-[4-hydroxy-3-(hydroxymethyl)phenyl]ethyl}amino)hexyl]oxy}butyl)phenyl]-N′-phenylurea acetate

was prepared using methods similar to those described in Example 1xi)

LCMS RT=2.63 min, ES+ve 550 (MH)⁺.

### Example 33

### N-[3-(4-{[6-({(2R)-2-Hydroxy-2-[4-hydroxy-3-(hydroxymethyl)phenyl]ethyl}amino)hexyl]oxy}butyl)phenyl]-N′-(3-hydroxyphenyl)urea

### i) N-(3-Hydroxyphenyl)-N′-(3-iodophenyl)urea

was prepared using methods similar to those described in Example 31i)

LCMS RT=3.39 min

### ii) N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}-N′-(3-hydroxyphenyl)urea

was prepared using methods similar to those described in Example 1vii)

LCMS RT=3.70 min

### iii) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}-N′-(3-hydroxyphenyl)urea

was prepared using methods similar to those described in Example 1viii)

LCMS RT=3.73 min

### iv) N-[3-(4-{[6-({(2R)-2-Hydroxy-2-[4-hydroxy-3-(hydroxymethyl)phenyl]ethyl}amino)hexyl]oxy}butyl)phenyl]-N′-(3-hydroxyphenyl)urea

was prepared using methods similar to those described in Example 14iii)

LCMS RT=2.59 min, ES+ve 566 (MH)⁺.

### Example 34

### [({[3-(4-{[6-({(2R)-2-Hydroxy-2-[4-hydroxy-3-(hydroxymethyl)phenyl]ethyl}amino)hexyl]oxy}butyl)phenyl]amino}carbonyl)amino](oxo)acetic acid

### i) N-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}urea

was prepared using methods similar to those described in Example 1 vii). LCMS RT=3.46 min.

### ii) N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}urea

was prepared using methods similar to those described in Example 1 viii). LCMS RT=3.37 min.

### iii) 1-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}imidazolidine-2,4,5-trione

N-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}urea (0.52 g) was dissolved in absolute ethanol (25 ml) and treated with diethyl oxalate (0.65 ml) and then sodium (0.07 g) in ethanol (7 ml). After stirring for 2 h another portion of sodium (0.023 g) in ethanol (2.3 ml) was added. After a further hour the reaction mixture was evaporated under reduced pressure and partitioned between pH 6.4 phosphate buffer and EtOAc. The organic layer was separated off and the aqueous phase extracted twice more with EtOAc. The combined extracts were dried (MgSO₄), evaporated under reduced pressure and purified by chromatography (Biotage, 40 g) eluting with EtOAc-cyclohexane (1:1) to give the title compound (0.277 g) LCMS RT=3.37 min.

### iv) ({[(3-{4-[(6-{[(2R)-2-(2,2-Dimethyl-4H-1,3-benzodioxin-6-yl)-2-hydroxyethyl]amino}hexyl)oxy]butyl}phenyl)amino]carbonyl}amino)(oxo)acetic acid

was prepared using methods similar to those described in Example 1x). LCMS RT=2.89 min.

### v) [({[3-(4-{[6-({(2R)-2-Hydroxy-2-[4-hydroxy-3-(hydroxymethyl)phenyl]ethyl}amino)hexyl]oxy}butyl)phenyl]amino}carbonyl)amino](oxo) acetic acid

was prepared using methods similar to those described in Example 1xi). LCMS RT=2.89 min, ES+ve 546 (MH)⁺.

### Example 35

### N²-({[3-(4-{[6-({(2R)-2-Hydroxy-2-[4-hydroxy-3-(hydroxymethyl)phenyl]ethyl}amino)hexyl]oxy}butyl)phenyl]amino}carbonyl)glycinamide formate

A solution of 3-[3-(4-{[6-({(2R)-2-hydroxy-2-[4-hydroxy-3-(hydroxymethyl)phenyl]ethyl}amino)hexyl]oxy}butyl)phenyl]imidazolidine-2,4-dione acetate (80 mg) (WO02070490A1) was dissolved in 2M ammonia in methanol solution and the mixture was stirred overnight at 20° C. The solvent was removed under reduced pressure and the residue was purified by mass directed autoprep to give the title compound (18.3 mg) LCMS RT=2.20 min, ES+ve m/z 531 (M+H)⁺.

### Example 36

### N¹-Cyclopentyl-N²-({[3-(4-{[6-({(2R)-2-hydroxy-2-[4-hydroxy-3-(hydroxymethyl)phenyl]ethyl}amino)hexyl]oxy}butyl)phenyl]amino}carbonyl)glycinamide acetate

A solution of 3-[3-(4-{[6-({(2R)-2-hydroxy-2-[4-hydroxy-3-(hydroxymethyl)phenyl]ethyl}amino)hexyl]oxy}butyl)phenyl]imidazolidine-2,4-dione acetate (240 mg) (WO02070490A1) was dissolved in ethanol (2 ml) and cyclopentylamine (3 ml) and the mixture was heated to 80° C. for 2 h and then allowed to cool to room temperature overnight. The solvent and excess amine were removed under reduced pressure and the residue was purified by chromatography on silica cartridge (10 g) eluting with a gradient of 1 to 10% methanol containing aqueous ammonia (1%) in dichloromethane. Appropriate fractions were evaporated to dryness and then dissolved in methanol (2 ml) and acetic acid (0.5 ml). The solution was evaporated to dryness under reduced pressure to give the title compound (115 mg) RT=2.62 min, ES+ve m/z 599 (M+H)⁺.

### Example 37

### N-(Aminocarbonyl)-N-[3-(4-{[6-({(2R)-2-hydroxy-2-[4-hydroxy-3-(hydroxymethyl)phenyl]ethyl}amino)hexyl]oxy}butyl)phenyl]- -alanine formate

### i) Ethyl N-(aminocarbonyl)-N-(3-iodophenyl)- -alaninate

A solution of 3-iodoaniline (1.2 g) in chloroform (5 ml) was treated with ethyl 3-bromopropionate (1.54 ml) and the mixture was stirred at room temperature for 19 h and then at 94° C. for 67 h. The solvent was evaporated under reduced pressure to give a mixture of starting material (39%), ethyl N-(3-iodophenyl)- -alaninate (29.5%) and dialkylated product (31.3%). LCMS RT=3.42 min, ES+ve m/z 320 (M+H)⁺. The reaction mixture was dissolved in acetic acid (4 ml), tetrahydrofuran (5 ml) and water (2 ml) and then treated with solid sodium cyanate (250 mg) and stirred for 22 h. The solvents were removed under reduced pressure and the residue was diluted with ethyl acetate and water. The organic phase washed with brine, dried (MgSO₄), and concentrated. The residue was triturated in dichloromethane-ether and the solid was removed by filtration. The filtrate was purified by chromatography on a Biotage cartridge (40 g) eluting with ether-cyclohexane (1:1) (500 ml), followed by 3% methanol-dichloromethane (500 ml) to give the title compound (0.4 g) LCMS RT=2.75 min, ES+ve m/z 362 (M+H)⁺.

### i) 1-(3-Iodophenyl)dihydropyrimidine-2,4(1H,3H)-dione

A solution of ethyl N-(aminocarbonyl)-N-(3-iodophenyl)- -alaninate (0.4 g) in tetrahydrofuran (4 ml) was treated with potassium trimethylsilanolate (160 mg) and the mixture was stirred for 18 h. The mixture was diluted with ethyl acetate and acidified with 2M hydrochloric acid. The organic layer washed with brine, dried (MgSO₄), and concentrated under reduced pressure to give the title compound (297 mg) LCMS RT=2.49 min, ES+ve m/z 317 (M+H)⁺.

### i) 1-{3-[4-({6-[(5R)-5-(2,2-Dimethyl-4H-1,3-benzodioxin-6-yl)-2-oxo-1,3-oxazolidin-3-yl]hexyl}oxy)but-1-ynyl]phenyl}dihydropyrimidine-2,4(1H,3H)-dione

was prepared using methods similar to those described in Example 1vii. LCMS RT=3.33 min, ES+ve m/z 590 (M+H)⁺.

### ii) 1-{3-[4-({6-[(5R)-5-(2,2-Dimethyl-4H-1,3-benzodioxin-6-yl)-2-oxo-1,3-oxazolidin-3-yl]hexyl}oxy)butyl]phenyl}dihydropyrimidine-2,4(1H,3H)-dione

was prepared using methods similar to those described in Example 1viii. LCMS RT=3.32 min, ES+ve m/z 594 (M+H)⁺

### v) N-(Aminocarbonyl)-N-(3-{4-[(6-{[(2R)-2-(2,2-dimethyl-4H-1,3-benzodioxin-6-yl)-2-hydroxyethyl]amino}hexyl)oxy]butyl}phenyl)- -alanine

was prepared using methods similar to those described in Example 1x. LCMS RT=2.47 min, ES+ve m/z 586 (M+H)⁺

### vi) N-(Aminocarbonyl)-N-[3-(4-{[6-({(2R)-2-hydroxy-2-[4-hydroxy-3-(hydroxymethyl)phenyl]ethyl}amino)hexyl]oxy}butyl)phenyl]- -alanine formate

was prepared using methods similar to those described in Example 1 xi) and purified by mass directed autoprep. LCMS RT=2.09 min, ES+ve m/z 546 (M+H)⁺

### Example 38

### N-[3-(4-{[6-({(2R)-2-Hydroxy-2-[4-hydroxy-3-(hydroxymethyl)phenyl]ethyl}amino)hexyl]oxy}butyl)-5-methylphenyl]urea

### i) 2-Bromo-4-methyl-6-nitroaniline

4-Methyl-2-nitroaniline (52.5 g) was suspended in glacial acetic acid (500 ml) and bromine (21.5 ml) added over 45 min at ambient temperature. The reaction mixture was stirred for 45 min, poured into water (3 L) and the suspension stirred for 30 min. The solid was filtered, washed with water and dried to give the title compound (72.7 g). ¹HNMR (CDCl₃, 400 MHz) ppm; 7.94 (1H, s), 7.56 (1H, s), 6.56 (2H, br s), 2.28 (3H, s).

### ii) 3-Bromo-5-nitrotoluene

2-Bromo-4-methyl-6-nitroaniline (20-5 g) was suspended in ethanol (105 ml) and sulfuric acid S.G. 1.84 (14 ml) added portionwise. The solution was heated to 73° C. and sodium nitrite (13.7 g) added over 25 min, maintaining the temperature at 73-78° C. for 30 min. The reaction mixture was cooled and then poured into water (700 ml). The solid was collected by filtration, washed with water and the product purified by steam distillation to give the title compound, (12.6 g). ¹HNMR (CDCl₃, 400 MHz) ppm; 8.19 (1H, br s) 7.98 (1H, brs), 7.66 (1H, brs), 2.46 (3H, s).

### iii) 6-Bromohexyl 4-(3-methyl-5-nitrophenyl)but-3-ynyl ether

3-Bromo-5-nitrotoluene (21.6 g) was dissolved in tetrahydrofuran (150 ml) and triethylamine (28.5 ml), copper (I) bromide (0.43 g), triphenylphosphine (0.55 g) and bis(triphenylphosphine) palladium (II) chloride (2.5 g) added and heated to 55° C. A solution of 6-bromohexyl but-3-ynyl ether (50 g) in tetrahydrofuran (150 ml) was added over 4 h. The mixture was cooled, the solvent was removed under reduced pressure and diethyl ether (100 ml) was added to the residue. The solid was collected by filtration and purified by silica gel column chromatography (20-50% dichloromethane-hexane) to give the title compound (18.5 g). ¹HNMR (CDCl₃, 400 MHz) ppm; 8.04 (1H, brs), 7.94 (1H, br s), 7.51 (1H, br s), 3.63 (2H, t, J 7 Hz), 3.50 (2H, t, J 7 Hz), 3.40 (2H, t, J 7 Hz), 2.70 (2H, t, J 7 Hz), 2.40 (3H, s), 1.86 (2H, m), 1.62 (2H, m), 1.45 (4H, m).

### iv) (1R)-2-[Benzyl(6-{[4-(3-methyl-5-nitrophenyl)but-3-ynyl]oxy}hexyl)amino]-1-(2,2-dimethyl-4H-1,3-benzodioxin-6-yl)ethanol

(1R)-2-(Benzylamino)-1-(2,2-dimethyl-4H-1,3-benzodioxin-6-yl)ethanol (16 g), N,N-diisopropylethylamine (21 ml), and 6-bromohexyl 4-(3-methyl-5-nitrophenyl)but-3-ynyl ether (18.9 g) were dissolved in acetonitrile (190 ml) and heated at reflux for 65 h. The mixture was cooled and partitioned between water and diethyl ether. The organic phase was separated, dried, the solvent evaporated and the residue purified by silica gel column chromatography (20-25% ethyl acetate-hexane) to give the title compound (20.3 g). ¹HNMR (CDCl₃, 400 MHz) ppm; 8.04 (1H, br s), 7.93 (1H, br s), 7.50 (1H, br s), 7.30 (5H, m), 7.04 (1H, dd, J 8, 2 Hz), 6.94 (1H, br s), 6.76 (1H, d, J 8 Hz), 4.82 (2H, s), 4.56 (1H, dd, J 4, 9 Hz), 3.88 (1H, d, J 13 Hz), 3.62 (2H, t, J 7 Hz), 3.47 (3H, m), 2.69 (2H, t, J 7 Hz), 2.67-2.40 (4H, m), 2.41 (3H, s), 1.68-1.48 (4H, m) 1.52 (6H, s), 1.40-1.23 (4H, m).

### v) (1R)-2-[{6-[4-(3-Amino-5-methylphenyl)butoxy]hexyl}(benzyl)amino]-1-(2,2-dimethyl-4H-1,3-benzodioxin-6-yl)ethanol

(1R)-2-[Benzyl(6-{[4-(3-methyl-5-nitrophenyl)but-3-ynyl]oxy}hexyl)amino]-1-(2,2-dimethyl-4H-1,3-benzodioxin-6-yl)ethanol (26.7 g) was dissolved in ethanol (260 ml) and hydrogenated over 5% platinum on carbon (2.7 g) at ambient temperature and atmospheric pressure, for 16 h. The catalyst was filtered and the solvent removed to give the title compound (23.6 g). ¹HNMR (CDCl₃, 400 MHz) ppm; 7.35-7.25 (5H, m), 7.04 (1H, dd, J 2, 8 Hz), 6.94 (1H, br s), 6.76 (1H, d, J 8 Hz), 6.42 (1H, br s), 6.33 (2H, br s), 4.82 (2H, s), 4.56, (1H, dd, J 4.9 Hz), 3.88 (1H, d, J 13 Hz), 3.49 (1H, d, J 13 Hz), 3.40 (2H, t, J 7 Hz), 3.36 (2H, t, J 7 Hz), 2.65-2.40 (6H, m), 2.22 (3H, s), 1.70-1.45 (8H, m), 1.53 (6H, s), 1.40-1.28 (4H, m).

### vi) N-(3-{4-[(6-{Benzyl[(2R)-2-(2,2-dimethyl-4H-1,3-benzodioxin-6-yl)-2-hydroxyethyl]amino}hexyl)oxy]butyl}-5-methylphenyl)urea

(1R)-2-[{6-[4-(3-Amino-5-methylphenyl)butoxy]hexyl}(benzyl)amino]-1-(2,2-dimethyl-4H-1,3-benzodioxin-6-yl)ethanol (58.2 g) was dissolved in glacial acetic acid (200 ml) and water (100 ml) and cooled to 0° C. A solution of potassium cyanate (17.2 g) in water (100 ml) was added over 10 min at 0-2° C. The mixture was stirred for 20 min, water (500 ml) added and the product extracted into dichloromethane. The organic phase was washed sequentially with saturated sodium bicarbonate, water and brine, dried and evaporated. The residue was purified by silica gel column chromatography (70-100% ethyl acetate-hexane) to give the title compound (33.5 g). ¹HNMR (CDCl₃, 400 MHz) ppm; 7.37-7.26 (5H, m), 7.06 (1H, dd, J 2, 8 Hz), 6.90 (1H, br s), 6.93 (1H, br s), 6.85 (1H, br s), 6.80-6.75 (3H, m), 4.80 (2H, s), 4.77 (2H, s), 4.57 (1H, dd, J 5.9 Hz), 3.89 (1H, d, J 13 Hz), 3.48 (1H, d, J 13 Hz), 3.41 (2H, t, J 7 Hz), 3.37 (2H, t, J 7 Hz), 2.67-2.41 (6H, m), 2.29 (3H, s), 1.80-1.48 (8H, m), 1.53 (6H, s), 1.39-1.28 (4H, m).

### vii) N-[3-(4-{[6-(Benzyl{(2R)-2-hydroxy-2-[4-hydroxy-3-(hydroxymethyl)phenyl]ethyl}amino)hexyl]oxy}butyl)-5-methylphenyl]urea

N-(3-{4-[(6-{Benzyl[(2R)-2-(2,2-dimethyl-4H-1,3-benzodioxin-6-yl)-2-hydroxyethyl]amino}hexyl)oxy]butyl}-5-methyl phenyl)urea (8.8 g) was dissolved in ethanol (80 ml) and 2M hydrochloric acid (25 ml) added and the reaction stirred at ambient temperature for 16 h. Saturated sodium bicarbonate (100 ml) was added and the product extracted into dichloromethane. The organic phase was dried and the solvent removed to give the title compound (7.88 g). ¹HNMR (CDCl₃, 400 MHz) ppm; 9.18 (1H, s), 8.39 (1H, s), 7.31-7.18 (6H, m), 7.03 (1H, s), 7.00 (1H, s), 6.94 (1H, dd, J 2, 8 Hz), 6.68 (1H, d, J 8 Hz), 6.53 (1H, s), 5.79 (2H, s), 4.95 (1H, t, J 5 Hz), 4.68 (1H, br), 4.56 (1H, br), 4.46 (2H, d, J 6 Hz), 3.61 (2H, m), 3.32 (2H, t, J 7 Hz), 3.27 (2H, t, J 7 Hz), 2.60-2.35 (7H, m), 2.20 (3H, s), 1.60-1.30 (8H, m), 1.22-1.10 (4H, m).

### viii) N-[3-(4-{[6-({(2R)-2-hydroxy-2-[4-hydroxy-3-(hydroxymethyl)phenyl]ethyl}amino)hexyl]oxy}butyl)-5-methylphenyl]urea

N-[3-(4-{[6-(Benzyl{(2R)-2-hydroxy-2-[4-hydroxy-3-(hydroxymethyl)phenyl]ethyl}amino)hexyl]oxy}butyl)-5-methylphenyl]urea (12.6 g) was dissolved in ethanol (120 ml) and hydrogenated over 10% palladium on carbon (2.4 g) at ambient temperature and atmospheric pressure for 16 h. The catalyst was filtered and the solvent removed to give the crude product (10.6 g). A portion of the crude product (5 g) was dissolved in hot ethanol (12 ml), cooled, 0.88 ammonia (1 ml) and chloroform (37 ml) added and the solution applied to a silica gel column, prepared and eluted with dichloromethane-ethanol-0.88 ammonia (25:8:1) to give the title compound (3.4 g). LC RT4.20 min.

From previous experiments this demonstrated that the product was the base corresponding to the compound of Example 29.

### Example 39

### N-[3-(4-{[6-({(2R)-2-Hydroxy-2-[4-hydroxy-3-(hydroxymethyl)phenyl]ethyl}amino)hexyl]oxy}butyl)-5-methylphenyl]urea

### i) N-(3-Bromo-5-methylphenyl)urea

To a solution of 3-bromo-5-nitrotoluene (1500 g) in glacial acetic acid (11 litres) in a nitrogen purged hydrogenation vessel was added 5% platinum on carbon (approx 50% water wet paste) and the mixture hydrogenated under 4 bar hydrogen pressure at room temperature. On completion of hydrogen uptake the catalyst was removed by filtration and the filtrate split into two equal portions. Each portion was set to stir separately and a solution of potassium cyanate (500 g) in water (1.25 litres) was added to each over 15 minutes. After stirring for an additional 15 minutes water (10 litres) was added and the precipitated solid isolated by filtration and washed with water (4 litres). The water wet cakes were combined and dissolved in hot ethyl acetate (3 litres) and the aqueous phase separated. The organic phase was cooled with stirring to crystallise the product, which was isolated by filtration and washed with fresh ethyl acetate (2 litres) and air dried overnight. Recrystallisation from ethanol (2.7 litres) afforded the title compound. (565 g) LC RT3.9 mins.

### ii) 6-Bromohexyl but-3-enyl ether

1,6-Dibromohexane (750 g) was added to a stirred solution of sodium hydroxide (375 g) in water (750 ml). Tetrabutylammonium bromide (6.5 g) was added and the two-phase mixture warmed to 50-55° C. 3-Buten-1-ol (150 g) was added over about 30 minutes and stirring continued at 50-55° C. for 4-6 hours. The mixture was cooled, diluted with tert-butyl methyl ether and the layers separated. The organic layer washed twice with water followed by brine and evaporated under vacuum to give the product as a liquid. This was purified by silica column chromatography, eluting initially with hexane then with 2.5% ethyl acetate in hexane. Product fractions were combined and evaporated to give the title compound (237 g). GC RT: 10.1 min.

### iii) N-(3-{4-[(6-Bromohexyl)oxy]butyl}-5-methylphenyl)urea

6-Bromohexylbut-3-enyl ether (80 g) was weighed into a nitrogen purged flask and a 0.5M solution of 9-BBN in THF (800 ml) added with stirring over 1-2 minutes. The resulting solution was left to stir at room temperature for 3 hours, then a solution of potassium phosphate (144 g) in water (204 ml) added. N-(3-Bromo-5-methylphenyl)urea) (74 g) was then added followed immediately by palladium acetate (0.8 g) and triphenylphosphine (1.8 g). The mixture was heated to 60° C. and maintained at this temperature for 1-4 hours until the reaction was complete. The mixture was cooled to room temperature and the layers separated. The organic layer washed with water and brine and evaporated to give the title compound as a residual oil (196 g) which was used directly at the next stage. LC RT 6.0 mins.

### iv) N-(3-{4-[(6-{Benzyl[(2R)-2-(2,2-dimethyl-4H-1,3-benzodioxin-6-yl)-2-hydroxyethyl]amino}hexyl)oxy]butyl}-5-methylphenyl)urea

To a stirred solution of N-(3-{4-[(6-bromohexyl)oxy]butyl}-5-methylphenyl)urea (equivalent of 40.7 g N-(3-bromo-5-methylphenyl)urea) in acetonitrile (200 ml) was added N,N-diisopropylethylamine (36.6 g) followed by (1R)-2-(benzylamino)-1-(2,2-dimethyl-4H-1,3-benzodioxin-6-yl)ethanol (WO02/066422) (53.4 g). The resulting mixture was heated to 65-75° C. and left to stir for 48 to 72 hours. The mixture was cooled, partitioned between water and dichloromethane and the layers separated. The organic layer washed with 1M HCl, water and brine and evaporated to an oil (147 g). The oil (2 g) was purified by silica column chromatography, eluting with ethyl acetate containing 1% aqueous ammonia solution to give the title compound as an oil (0.95 g). LC RT 4.9 mins.

### v) N-[3-(4-{[6-({(2R)-2-Hydroxy-2-[4-hydroxy-3-(hydroxymethyl)phenyl]ethyl}amino)hexyl]oxy}butyl)-5-methylphenyl]urea

The product of Example 39 iv) may be deprotected as in Example 38 vii) and viii).

### Examples 40-42

### Preparation of salts of N-[3-(4-{[6-({(2R)-2-hydroxy-2-[4-hydroxy-3-(hydroxymethyl)phenyl]ethyl}amino)hexyl]oxy}butyl)-5-methylphenyl]urea

### Example 40

L-Aspartate salt: A hot solution of the N-[3-(4-{[6-({(2R)-2-hydroxy-2-[4-hydroxy-3-(hydroxymethyl)phenyl]ethyl}amino)hexyl]oxy}butyl)-5-methylphenyl]urea (500 mg) in ethanol (5 ml) was added to a hot solution of L-aspartic acid (136.5 mg) in water (5 ml) to give a solution of the salt. This was evaporated to an oil which was dissolved in a mixture of ethanol (5 ml) and water (1 ml). Dichloromethane (10 ml) was added and the cloudy solution left to stir overnight. The resulting solid was filtered, washed with a mixture of ethanol (0.65 ml) and dichloromethane (1.3 ml) and air dried to give the title compound (443 mg).

δ (DMSO-d₆) 8.74 (1H, s), 7.29 (1H, s), 7.10 (1H, s), 7.02 (2H, m), 6.73 (1H, d, J 8.3 Hz), 6.52 (1H, s), 5.95 (2H, s), 4.67 (1H, m), 4.48 (2H, s), 3.53 (1H, t, J 6.6 Hz), 3.34 (4H, m), 2.81-2.71 (4H, m), 2.58 (1H, dd, J 7.8 Hz, 16.1 Hz), 2.46 (2H, t, J 7.1 Hz), 2.32 (1H, dd, J 6.4 Hz, 16.1 Hz), 2.20 (3H, s), 1.58-1.48 (8H, m), 1.30 (4H, m).

### Example 41

Triphenylacetate salt: N-[3-(4-{[6-({(2R)-2-hydroxy-2-[4-hydroxy-3-(hydroxymethyl)phenyl]ethyl}amino)hexyl]oxy}butyl)-5-methylphenyl]urea (500 mg) and triphenylacetic acid (295.7 mg) were dissolved in hot ethanol (5 ml). Water (5 ml) was added causing a gum to separate. The mixture was stirred overnight forming a solid suspension which was filtered, washed with aqueous ethanol and dried at 50° C. under vacuum, to give the title compound (543 mg).

δ (CD₃OD) 7.30-7.09 (18H, m), 7.01 (1H, s), 6.94 (1H, s), 6.76 (1H, d, J 8.3 Hz), 6.64 (1H, s), 4.64 (2H, s), 3.40 (4H, m), 2.99 (2H, m), 2.88 (2H, t, J 8.1 Hz), 2.52 (2H, t, J 7.1 Hz), 2-23 (3H, s), 1.68-1.51 (8H, m), 1.30 (4H, m)

### Example 42

1-naphthoate salt: N-[3-(4-{[6-({(2R)-2-hydroxy-2-[4-hydroxy-3-(hydroxymethyl)phenyl]ethyl}amino)hexyl]oxy}butyl)-5-methylphenyl]urea (500 mg) and 1-naphthoic acid (176.6 mg) were dissolved in hot ethanol (5 ml). Water (5 ml) was added and the solution left to stir and cool overnight to precipitate the salt. The solid was isolated by filtration, washed with aqueous ethanol and dried at 50° C. under vacuum, to give the title compound (402 mg).

δ (DMSO-d₆) 8.94 (1H, broad d, J 6.6 Hz), 8.74 (1H, s), 7.91 (3H, broad d, J 7.1 Hz), 7.49 (3H, m), 7.34 (1H, s), 7.05 (3H, broad d, J 6.6 Hz), 6.76 (1H, d, J 8.1 Hz), 6.52 (1H, s), 5.95 (2H, s), 4.83 (1H, broad d, J 8.1 Hz), 4.49 (2H, s), 3.31 (4H, m), 2.98-2.84 (4H, m), 2.45 (1H, t, J 7.1 Hz), 2.20 (3H, s), 1.61-1.47 (8H, m), 1.30 (4H, m).

Biological Activity

The potencies of the aforementioned compounds were determined using frog melanophores transfected with the human beta 2 adrenoreceptor. The cells were incubated with melatonin to induce pigment aggregation. Pigment dispersal was induced by compounds acting on the human beta 2 adrenoreceptor. The beta 2 agonist activity of test compounds was assessed by their ability to induce a change in light transmittance across a melanophore monolayer (a consequence of pigment dispersal). At the human beta 2 adrenoreceptor, compounds of examples 1-37 had IC₅₀ values below 1 μM.

Potency at other beta adrenoreceptor subtypes was determined using chinese hamster ovary cells transfected with either the human beta 1 adrenoreceptor or the human beta 3 adrenoreceptor. Agonist activity was assessed by measuring changes in intracellular cyclic AMP.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 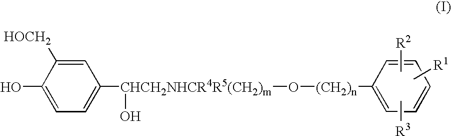
or a salt, solvate, or physiologically functional derivative thereof, wherein:
m is an integer of from 2 to 8;
n is an integer of from 3 to 11;
with the proviso that m+n is 5 to 19;
R¹ is —XNR⁶C(O)NR⁷R⁸; wherein
X is selected from —(CH₂)ₚ— and C₂₋₆alkenylene;
R⁶ and R³ are independently selected from hydrogen, C₁₋₆alkyl and C₃₋₇ cycloalkyl;
R⁷ is selected from hydrogen, C₁₋₆alkyl, C₃₋₇cycloalkyl, —C(O)R⁹, phenyl, naphthyl, hetaryl, and phenyl(C₁₋₄alkyl)- and R⁷ is optionally substituted by 1 or 2 groups independently selected from halo, hydroxy, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H, —CO₂(C₁₋₄alkyl) and CONR¹⁰R¹¹;
R⁹ is selected from C₁₋₆alkyl, C₃₋₇cycloalkyl, —CO₂H, CO₂(C₁₋₄alkyl), phenyl, naphthyl, hetaryl, and phenyl(C₁₋₄alkyl)- and R⁹ is optionally substituted by 1 or 2 groups independently selected from halo, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H,
—CO₂(C₁₋₄alkyl);
R¹⁰ and R¹¹ each independently represent hydrogen,
C₁₋₄alkyl or C₃₋₇ cycloalkyl, and
p is an integer from 0 to 6;
or R¹ is cyclised such that R⁸ forms a bond with the phenyl ring to which R¹ is attached, via the ring carbon atom adjacent to R¹, so as to form a moiety of the formula: 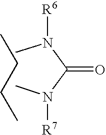
R² is selected from hydrogen, C₁₋₆alkyl, C₁₋₆alkoxy, phenyl, halo, and C₁₋₆haloalkyl;
R³ is selected from hydrogen, hydroxy, C₁₋₆alkyl, halo, C₁₋₆ alkoxy, phenyl, C₁₋₆haloalkyl, and —SO₂NR¹²R¹³;
wherein R¹² and R¹³ are independently selected from hydrogen, C₁₋₆alkyl, C₃₋₆cycloalkyl, phenyl, and phenyl (C₁₋₄alkyl), or R¹² and R¹³, together with the nitrogen to which they are bonded, form a 5-, 6-, or 7-membered nitrogen containing ring;
and R¹² and R¹³ are each optionally substituted by one or two groups selected from halo, C₁₋₆alkyl, and C₁₋₆haloalkyl;
R⁴ and R⁵ are independently selected from hydrogen and C₁₋₄alkyl with the proviso that the total number of carbon atoms in R⁴ and R⁵ is not more than 4;
with the provisos that:
a) when R², R³, R⁴, R⁵, and R⁶ each denote hydrogen, m is 5, n is 2, and R¹ denotes —(CH₂)ₚ— and is in the para position relative to the —O—(CH₂)ₙ— link, and p is 0, then R⁷ and R⁸ are not both hydrogen; and
b) when R², R³, R⁴, R⁵, and R⁶ each denote hydrogen, m is 5, n is 4, and R¹ denotes —(CH₂)ₚ— and is in the para position relative to the —O—(CH₂)ₙ— link, and p is 0, then R⁷ and R⁸ are not both methyl.

2. A compound of formula (Ia) 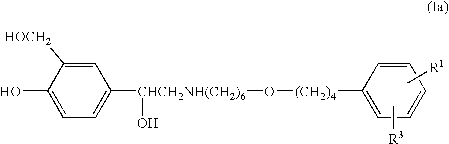
or a salt, solvate, or physiologically functional derivative thereof,
wherein:
R¹ is —XNR⁶C(O)NR⁷R⁸; wherein
X is selected from —(CH₂)ₚ— and C₂₋₆alkenylene;
R⁵ and R⁸ are independently selected from hydrogen, C₁₋₆alkyl and C₃₋₇ cycloalkyl;
R⁷ is selected from hydrogen, C₁₋₆alkyl C₃₋₇cycloalkyl, —C(O)R⁹, phenyl, naphthyl, hetaryl, and phenyl(C₁₋₄alkyl)- and R⁷ is optionally substituted by 1 or 2 groups independently selected from halo, hydroxy, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H, —CO₂(C₁₋₄alkyl and CONR¹⁰R¹¹;
R⁹ is selected from C₁₋₆alkyl, C₃₋₇cycloalkyl, —CO₂H, CO₂(C₁₋₄alkyl), phenyl, naphthyl, hetaryl, and phenyl(C₁₋₄alkyl)- and R⁹ is optionally substituted by 1 or 2 groups independently selected from halo, C₁₋₆alkyl, C₁₋₆haloalkyl, C₁₋₆ alkoxy, —NHC(O)(C₁₋₆alkyl), —SO₂(C₁₋₆alkyl), —SO₂(phenyl), —CO₂H,
—CO₂(C₁₋₄alkyl);
R¹⁰ and R¹¹ each independently represent hydrogen,
C₁₋₄alkyl Or C₃₋₇ cycloalkyl and
p is an integer from 0 to 6;
or R¹ is cyclised such that R⁸ forms a bond with the phenyl ring to which R¹ is attached, via the ring carbon atom adjacent to R¹, so as to form a moiety of the formula: 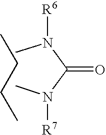
and R³ is selected from hydrogen, hydroxy, C₁₋₆alkyl, halo, C₁₋₆alkoxy, phenyl, C₁₋₆haloalkyl, and —SO₂NR¹²R¹³;
wherein R¹² and R¹³ are independently selected from hydrogen, C₁₋₆alkyl C₃₋₆cycloalkyl, phenyl, and phenyl (C₁₋₄alkyl), or R¹² and R¹³, together with the nitrogen to which they are bonded, form a 5-, 6-, or 7-membered nitrogen containing ring;
and R¹² and R¹³ are each optionally substituted by one or two groups selected from halo, C₁₋₆alkyl, and C₁₋₆haloalkyl.

3. A compound of formula (Ib) 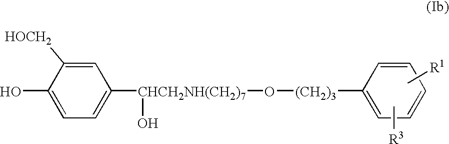
or a salt, solvate, or physiologically functional derivative thereof, wherein:
R¹ is —XNR⁶C(O)NR⁷R⁸; wherein
X is selected from —(CH₂)ₚ— and C₂₋₆alkenylene;
R⁶ and R⁸ are independently selected from hydrogen, C₁₋₆alkyl and C₃₋₇ cycloalkyl;
R⁷ is selected from hydrogen, C₁₋₆alkyl, C₃₋₇cycloalkyl, —C(O)R⁹, phenyl, naphthyl, hetaryl, and phenyl(C₁₋₄alkyl)- and R⁷ is optionally substituted by 1 or 2 groups independently selected from halo, hydroxy, C₁₋₆alkyl, C₁₋₆haloalkyl, C₁₋₆ alkoxy, —NHC(O)(C₁₋₆alkyl), —SO₂(phenyl), —CO₂H, —CO₂(C₁₋₄alkyl) and CONR¹⁰R¹¹;
R⁹ is selected from C₁₋₆alkyl, C₃₋₇cycloalkyl, —CO₂H, CO₂(C₁₋₄alkyl), phenyl, naphthyl, hetaryl, and phenyl(C₁₋₄alkyl)- and R⁹ is optionally substituted by 1 or 2 groups independently selected from halo, C₁₋₆alkyl C₁₋₆haloalkyl, C₁₋₆ alkoxy, —NHC(O)(C₁₋₆alkyl), —SO₂(C₁₋₆alkyl), —SO₂(phenyl), —CO₂H
—CO₂(C₁₋₄alkyl);
R¹⁰ and R¹¹ each independently represent hydrogen,
C₁₋₄alkyl or C₃₋₇ cycloalkyl, and
p is an integer from 0 to 6;
or R¹ is cyclised such that R⁸ forms a bond with the phenyl ring to which R¹ is attached, via the ring carbon atom adjacent to R¹, so as to form a moiety of the formula: 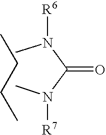
and R³ is selected from hydrogen, hydroxy, C₁₋₆alkyl, halo, C₁₋₆ alkoxy, phenyl, C₁₋₆haloalkyl, and —SO₂NR¹²R¹³;
wherein R¹² and R¹³ are independently selected from hydrogen, C₁₋₆alkyl, C₃₋₆cycloalkyl, phenyl, and phenyl (C₁₋₄alkyl), or R¹² and R¹³, together with the nitrogen to which they are bonded, form a 5-, 6-, or 7-membered nitrogen containing ring;
and R¹² and R¹³ are each optionally substituted by one or two groups selected from halo, C₁₋₆alkyl, and C₁₋₆haloalkyl.

4. A compound of formula (I), (Ia) or (Ib) selected from:
N-[3-(4-{[6-({(2R)-2-hydroxy-2-[4-hydroxy-3-(hydroxymethyl)phenyl]ethyl}amino)hexyl]oxy}butyl)phenyl]urea;
3-(4-{[6-({(2R)-2-hydroxy-2-[4-hydroxy-3-(hydroxymethyl)phenyl]ethyl}-amino)hexyl]oxy}butyl)phenyl)-N′-phenylurea;
N-[3-(4-{[6-({(2S)-2-hydroxy-2-[4-hydroxy-3-(hydroxymethyl)phenyl]ethyl}amino)hexyl]oxy}butyl)phenyl]urea;
3-(4-{[6-({(2S)-2-hydroxy-2-[4-hydroxy-3-(hydroxymethyl)phenyl]ethyl}-amino)hexyl]oxy}butyl)phenyl)-N′-phenylurea;
N-[3-(4-{[6-({2-hydroxy-2-[4-hydroxy-3-(hydroxymethyl)phenyl]ethyl}amino)hexyl]oxy}butyl)phenyl]urea;
3-(4-{[6-({(2-hydroxy-2-[4-hydroxy-3-(hydroxymethyl)phenyl]ethyl}-amino)hexyl]oxy}butyl)phenyl)-N′-phenylurea; and
N-[3-(4-{[6-({(2R)-2-hydroxy-2-[4-hydroxy-3-(hydroxymethyl)phenyl]ethyl}amino)hexyl]oxy}butyl)-5-methylphenyl]urea;
or a salt, solvate or physiologically functional equivalent thereof.

5. N-[3-(4-{[6-({(2R)-2-hydroxy-2-[4-hydroxy-3-(hydroxymethyl)phenyl]ethyl}amino)hexyl]oxy}butyl)-5-methylphenyl]urea;
or a salt or solvate thereof.

6. A method for the prophylaxis or treatment of a clinical condition in a mammal, such as a human, for which a selective 2-adrenoreceptor agonist is indicated, which comprises administration of a therapeutically effective amount of a compound according to any of claims 1 to 5, or a pharmaceutically acceptable salt, solvate, or physiologically functional derivative thereof.

7. (canceled)

8. A pharmaceutical formulation comprising a compound according to any of claims 1 to 5 or a pharmaceutically acceptable salt, solvate, or physiologically functional derivative thereof, and a pharmaceutically acceptable carrier or excipient, and optionally one or more other therapeutic ingredients.

9. (canceled)

10. A combination comprising a compound according to any of claims 1 to 5 or a pharmaceutically acceptable salt, solvate, or physiologically functional derivative thereof, and one or more other therapeutic ingredients.

11. A combination according to claim 10 wherein the other therapeutic ingredient is a corticosteroid, an anticholinergic or a PDE4 inhibitor.

12. A process for the preparation of a compound according to any of claims 1 to 5, or a salt, solvate, or physiologically functional derivative thereof, which comprises forming said compound by a reaction scheme selected from the group consisting of (a), (b), (c), (d), (e) and (f):
(a) deprotecting a protected intermediate of formula (II): 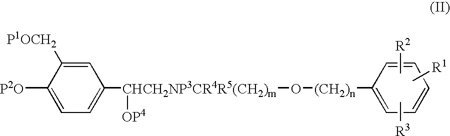
or a salt or solvate thereof, wherein R¹, R², R³, R⁴, R⁵, m, and n are as defined for the compound of formula (I), (Ia) or (Ib), and P¹, P², P³ and P⁴ are each independently either hydrogen or a protecting group provided that at least one of P¹, P², P³ and P⁴ is a protecting group;
(b) alkylating an amine of formula (XII) 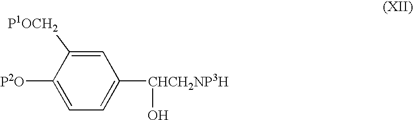
wherein P¹, P² and P³ are each independently either hydrogen or a protecting group,
with a compound of formula (XIII): 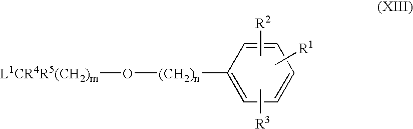
wherein R¹, R², R³, R⁴, R⁵, m, and n are as defined for the compound of formula (I) or (Ia) and L¹ is a leaving group;
(c) reducing a compound of formula (XV): 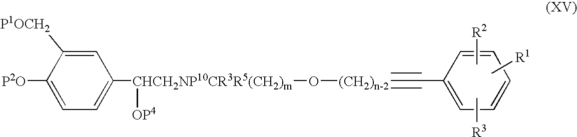
wherein R¹, R², R³, R⁴, R⁵, m and n are as defined for formula (I) and P¹, P², P³ and P⁴ are each independently hydrogen or a protecting group as defined above;
(d) reacting a compound of formula (XIX): 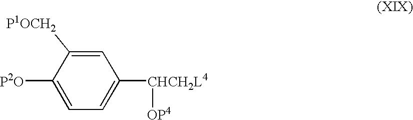
wherein P¹, P² and P⁴ are as hereinbefore defined and L⁴ is a leaving group as defined above for groups L-L³ with an amine of formula (XX): 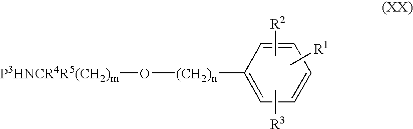
wherein R¹, R², R³, R⁴, R⁵, P³, m and n are as defined for formula (II);
(e) removing a chiral auxiliary from a compound of formula (IIa): 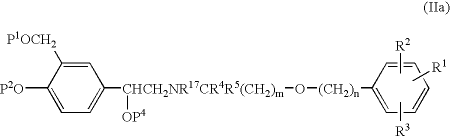
wherein R¹-R⁵, m and n are as defined for formula (I), P¹, P² and P⁴ each independently represent hydrogen or a protecting group and R¹⁷ represents a chiral auxiliary; and
(f) reacting a compound of formula (XXIII):
with an amine HNR¹⁰R¹¹; 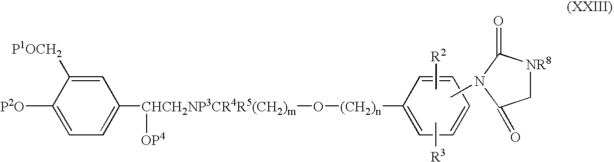
wherein P¹, P², P³, P⁴, R², R³, R⁴, R⁵ and R⁸ are as defined above,
with an amine of formula HNR¹⁰R¹¹,
wherein R¹⁰ and R¹¹ are as hereinbefore defined,
wherein any of (a), (b), (c), (d), (e), or (f) may optionally include one or more steps in any order selected from the group consisting of
(i) removing any protecting groups;
(ii) separating an enantiomer from a mixture of enantiomers; and
(iii) converting the product to a corresponding salt, solvate, or physiologically functional derivative thereof.
